(19) United States
(12) Patent Application Publication
(10) Pub. No.: US 2015/0122685 A1
(43) Pub. Date: May 7, 2015

(54) PACKAGING SYSTEM FOR PATIENT INTERFACE SYSTEM

(71) Applicant: ResMed Limited, Bella Vista (AU)

(72) Inventors: Dale Alan Stuart WAKEHAM, Sydney (AU); Martin Kang, Sydney (AU); Andrew James Bate, Blue Mountains (AU); Frederick Arlet May, Sydney (AU); Murray William Lee, Sydney (AU); Hadley White, Sydney (AU)

(73) Assignee: ResMed Limited, Bella Vista (AU)

(21) Appl. No.: 14/074,139

(22) Filed: Nov. 7, 2013

(51) Int. Cl. A62B 25/00; B65D 1/36; B65D 25/04; A61M 16/06; A61M 16/08

(52) U.S. Cl. CPC A62B 25/00; A61M 16/0622; A61M 16/0875; B65D 25/04; B65D 1/36
USPC 206/438; 53/474

(57) ABSTRACT

A packaging tray for a patient interface system, the patient interface system adapted to provide respiratory therapy, the packaging tray comprising: at least one cushion assembly region, each shaped to conform to at least a portion of a cushion assembly; a tube region shaped to conform to at least a portion of a tube; and a positioning and stabilising structure region shaped to conform to at least a portion of a positioning and structure.

### 1 BACKGROUND OF THE TECHNOLOGY

### 1.1 Field of the Technology

The present technology relates to one or more of the diagnosis, treatment and amelioration of respiratory disorders, and to procedures to prevent respiratory disorders. In particular, the present technology relates to medical devices, and their use for treating respiratory disorders and for preventing respiratory disorders.

### 1.2 Description of the Related Art

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the air into the venous blood and carbon dioxide to move out. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See West, Respiratory Physiology—the essentials.

A range of respiratory disorders exist.

Obstructive Sleep Apnea (OSA), a form of Sleep Disordered Breathing (SDB), is characterized by occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is a disorder of a patient's respiratory controller in which there are rhythmic alternating periods of waxing and waning ventilation, causing repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

Otherwise healthy individuals may take advantage of systems and devices to prevent respiratory disorders from arising.

### 1.3 Systems

One known product used for treating sleep disordered breathing is the S9 Sleep Therapy System, manufactured by ResMed. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit.

### 1.3.1 Therapy

Nasal Continuous Positive Airway Pressure (CPAP) therapy has been used to treat Obstructive Sleep Apnea (OSA). The hypothesis is that continuous positive airway pressure acts as a pneumatic splint and may prevent upper airway occlusion by pushing the soft palate and tongue forward and away from the posterior oropharyngeal wall.

Non-invasive ventilation (NIV) provides ventilator support to a patient through the upper airways to assist the patient in taking a full breath and/or maintain adequate oxygen levels in the body. The ventilator support is provided by a mask or nasal interface. NIV has been used to treat OHS, COPD, MD and Chest Wall disorders.

Invasive ventilation (IV) provides ventilatory support to patient's that are no longer able to effectively breathe themselves and is provided using a tracheotomy tube.

Ventilators also control the timing and pressure of breaths pumped into the patient and monitor the breaths taken by the patient. The methods of control and monitoring patients typically include volume-cycled and pressure-cycled methods. The volume-cycled methods may include among others, Pressure-Regulated Volume Control (PRVC), Volume Ventilation (VV), and Volume Controlled Continuous Mandatory Ventilation (VC-CMV) techniques. The pressure-cycled methods may involve, among others, Assist Control (AC), Synchronized Intermittent Mandatory Ventilation (SIMV), Controlled Mechanical Ventilation (CMV), Pressure Support Ventilation (PSV), Continuous Positive Airway Pressure (CPAP), or Positive End Expiratory Pressure (PEEP) techniques.

### 1.3.2 Patient Interface

The application of a supply of air at positive pressure to the entrance of the airways of a patient is facilitated by the use of a patient interface, such as a nasal mask, full-face mask or nasal pillows. A range of patient interface devices are known, however a number of them suffer from being one or more of obtrusive, aesthetically undesirable, poorly fitting, difficult to use and uncomfortable especially when worn for long periods of time or when a patient is unfamiliar with a system. Masks designed solely for aviators, as part of personal protection equipment or for the administration of anaesthetics may be tolerable for their original application, but nevertheless be undesirably uncomfortable to be worn for extended periods, for example, while sleeping or throughout the day

A tracheotomy tube is another form of patient interface that may be used for invasive ventilation.

### 1.3.2.1 Seal-Forming Portion

Patient interfaces typically include a seal-forming portion.

One type of seal-forming portion extends around the periphery of the patient interface, and is intended to seal against the user's face when force is applied to the patient interface with the seal-forming portion in confronting engagement with the user's face. The seal-forming portion may include an air or fluid filled cushion, or a moulded or formed surface of a resilient seal element made of an elastomer such as a rubber. With this type of seal-forming portion, if the fit is not adequate, there will be gaps between the seal-forming portion and the face, and additional force will be required to force the patient interface against the face in order to achieve a seal.

Another type of seal-forming portion incorporates a flap seal of thin material so positioned about the periphery of the mask so as to provide a self-sealing action against the face of the user when positive pressure is applied within the mask. Like the previous style of seal forming portion, if the match between the face and the mask is not good, additional force may be required to effect a seal, or the mask may leak. Furthermore, if the shape of the seal-forming portion does not match that of the patient, it may crease or buckle in use, giving rise to leaks.

Another form of seal-forming portion may use adhesive to effect a seal. Some patients may find it inconvenient to constantly apply and remove an adhesive to their face.

A range of patient interface seal-forming portion technologies are disclosed in the following patent applications, assigned to ResMed Limited: WO 1998/004,310; WO 2006/074,513; WO 2010/135,785.

### 1.3.2.2 Positioning and Stabilising

A seal-forming portion of a patient interface used for positive air pressure therapy is subject to the corresponding force of the air pressure to disrupt a seal. Thus a variety of techniques have been used to position the seal-forming portion, and to maintain it in sealing relation with the appropriate portion of the face.

One technique is the use of adhesives. See for example US Patent publication US 2010/0000534.

Another technique is the use of one or more straps and stabilising harnesses. Many such harnesses suffer from being one or more of ill-fitting, bulky, uncomfortable and awkward to use.

### 1.3.2.3 Vent Technologies

Some forms of patient interface systems may include a vent to allow the washout of exhaled carbon dioxide. Many such vents are noisy. Others may block in use and provide insufficient washout. Some vents may be disruptive of the sleep of a bed-partner 1100 of the patient 1000, e.g. through noise or focussed airflow.

ResMed Limited has developed a number of improved mask vent technologies. See WO 1998/034,665; WO 2000/078,381; U.S. Pat. No. 6,581,594; US Patent Application; US 2009/0050156; US Patent Application 2009/0044808.

Table of noise of prior masks
(ISO 17510-2:2007, 10 cm H₂O pressure at 1 m)

| | | A-weighted | A-weighted | |
|---|---|---|---|---|
| | | sound power | sound pressure | |
| | | level dbA | dbA | Year |
| Mask name | Mask type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| Mirage (*) | | | | |
| ResMed | nasal | 36 (3) | 28 (3) | 2000 |
| UltraMirage | | | | |
| ResMed | nasal | 32 (3) | 24 (3) | 2002 |
| Mirage Activa | | | | |
| ResMed | nasal | 30 (3) | 22 (3) | 2008 |
| Mirage Micro | | | | |
| ResMed | nasal | 29 (3) | 22 (3) | 2008 |
| Mirage SoftGel | | | | |
| ResMed | nasal | 26 (3) | 18 (3) | 2010 |
| Mirage FX | | | | |
| ResMed | nasal pillows | 37 | 29 | 2004 |
| Mirage Swift | | | | |
| (*) | | | | |
| ResMed | nasal pillows | 28 (3) | 20 (3) | 2005 |
| Mirage Swift II | | | | |
| ResMed | nasal pillows | 25 (3) | 17 (3) | 2008 |
| Mirage Swift | | | | |
| LT | | | | |

(* one specimen only, measured using test method specified in IS03744 in CPAP mode at 10 cm H₂O)

Sound pressure values of a variety of objects are listed below

| | A-weighted sound | |
|---|---|---|
| | pressure dbA | |
| Object | (uncertainty) | Notes |
| Vacuum cleaner: Nilfisk | 68 | ISO3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 1.3.2.4 Nasal Pillow Technologies

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT nasal pillows mask, SWIFT II nasal pillows mask, SWIFT LT nasal pillows mask, SWIFT FX nasal pillows mask and LIBERTY full-face mask. The following patent applications, assigned to ResMed Limited, describe nasal pillows masks: International Patent Application WO2004/073,778 (describing amongst other things aspects of ResMed SWIFT nasal pillows), US Patent Application 2009/0044808 (describing amongst other things aspects of ResMed SWIFT LT nasal pillows); International Patent Applications WO 2005/063,328 and WO 2006/130,903 (describing amongst other things aspects of ResMed LIBERTY full-face mask); International Patent Application WO 2009/052,560 (describing amongst other things aspects of ResMed SWIFT FX nasal pillows).

### 1.3.3 PAP Device

The air at positive pressure may be supplied to the airway of a patient by a PAP device such as a motor-driven blower. The outlet of the blower is connected via a flexible delivery conduit to a patient interface as described above.

Ventilators typically include a flow generator, an inlet filter, a patient interface, an air delivery conduit connecting the flow generator to the patient interface, various sensors and a microprocessor-based controller. The patient interface may include a mask or a tracheotomy tube as described above. The flow generator may include a servo-controlled motor, volute and an impeller that forms a blower. In some cases a brake for the motor may be implemented to more rapidly reduce the speed of the blower so as to overcome the inertia of the motor and impeller. The braking can permit the blower to more rapidly achieve a lower pressure condition in time for synchronization with expiration despite the inertia. In some cases the flow generator may also include a valve capable of discharging generated air to atmosphere as a means for altering the pressure delivered to the patient as an alternative to motor speed control. The sensors measure, amongst other things, motor speed, mass flow rate and outlet pressure, such as with a pressure transducer or the like. The apparatus may optionally include a humidifier and/or heater elements in the path of the air delivery circuit. The controller may include data storage capacity with or without integrated data retrieval and display functions.

### 1.3.4 Humidifier

Respiratory apparatuses commonly have the ability to alter the humidity of the breathable gas in order to reduce drying of the patient's airway and consequent patient discomfort and associated complications. The use of a humidifier placed between the flow generator or PAP device or ventilator and the patient interface produces humidified gas that minimizes drying of the nasal mucosa and increases patient airway comfort. In addition in cooler climates, warm air applied generally to the face area in and about the patient interface is more comfortable than cold air.

Humidity refers to the quantity of water vapour present in the air. It is commonly measured in two ways:
(1) Absolute Humidity (AH) is the actual content of water recorded in terms of weight per volume—usually in grams per cubic meter (g/m3) or milligrams per liter (mg/L).
(2) Relative Humidity (RH) is a percentage expression of the actual water vapour content of a gas compared to its capacity to carry water at any given temperature.

The capacity of air to hold water vapour increases as the temperature of the air increases. This means that for air with a stable AH, the RH will decline as the temperature of the air is increased. Conversely, for air saturated with water (100% RH), if the temperature is reduced then the excess water will condense out. Air breathed by humans is generally naturally heated and humidified by the airway to reach a temperature of 37° C. and 100% humidity. At this temperature the AH humidity is 44 mg/L.

Respiratory humidifiers are available in many forms and may be a standalone device that is coupled to a respiratory device via a air delivery tube, is integrated with the respiratory device or configured to be directly coupled to the relevant respiratory apparatus. While passive humidifiers can provide some relief, generally a heated humidifier is required to provide sufficient humidity and temperature to the air so that the patient will be comfortable. Humidifiers typically comprise a water reservoir or tub having a capacity of several hundred milliliters (ml), a heating element for heating the water in the reservoir, a control to enable the level of humidification to be varied, a gas inlet to receive gas from the flow generator or PAP device, and a gas outlet adapted to be connected to an air delivery conduit that delivers the humidified gas to the patient interface.

Heated passover humidification is one common form of humidification used with a PAP device. In such systems the heating element may be incorporated in a heater plate which sits under, and is in thermal contact with, the water tub. Thus, heat is transferred from the heater plate to the water reservoir primarily by conduction. The air flow from the PAP device or flow generator or ventilator passes over the heated water in the water tub resulting in water vapour being taken up by the air flow. The ResMed H4i™ and H5i™ Humidifiers are examples of such heated passover humidification systems that are used in combination with ResMed S8 and S9 CPAP systems respectively.

Other humidification systems may also be used such as a bubble or diffuser humidifier, a jet humidifier or a wicking humidifier. In a bubble or diffuser humidifier the air is conducted below the surface of the water and allowed to bubble back to the top. A jet humidifier produces an aerosol of water and baffles or filters may be used so that the particles are either removed or evaporated before leaving the humidifier. A wicking humidifier uses a water absorbing material, such as sponge or paper, to absorb water by capillary action. The water absorbing material is placed within or adjacent at least a portion of the air flow path to allow evaporation of the water in the absorbing material to be taken up into the air flow.

An alternative form of humidification is provided by the ResMed HumiCare™ D900 humidifier that uses a CounterStream™ technology that directs the air flow over a large surface area in a first direction whilst supplying heated water to the large surface area in a second opposite direction. The ResMed HumiCare™ D900 humidifier may be used with a range of invasive and non-invasive ventilators.

### 1.3.5 Mandibular Repositioning

A mandibular repositioning device (MRD) or mandibular advancement device (MAD) is one of the treatment options for sleep apnea and snoring. It is an adjustable oral appliance available from a dentist or other supplier that holds the lower jaw (mandible) in a forward position during sleep. The MRD is a removable device that a patient inserts into their mouth prior to going to sleep and removes following sleep. Thus, the MRD is not designed to be worn all of the time. The MRD may be custom made or produced in a standard form and include a bite impression portion designed to allow fitting to a patient's teeth. This mechanical protrusion of the lower jaw expands the space behind the tongue, puts tension on the pharyngeal walls to reduce collapse of the airway and diminishes palate vibration.

In certain examples a mandibular advancement device may comprise an upper splint that is intended to engage with or fit over teeth on the upper jaw or maxilla and a lower splint that is intended to engage with or fit over teeth on the upper jaw or mandible. The upper and lower splints are connected together laterally via a pair of connecting rods. The pair of connecting rods are fixed symmetrically on the upper splint and on the lower splint.

In such a design the length of the connecting rods is selected such that when the MRD is placed in a user's mouth the mandible is held in an advanced position. The length of the connecting rods may be adjusted to change the level of protrusion of the mandible. A dentist may determine a preferred level of protrusion for the mandible that will determine the length of the connecting rods

Some MRDs are structured to push that mandible forward relative to the maxilla while other MADs, such as the ResMed Narval CC™ MRD are designed to retain the mandible in a forward position. This device also reduces or minimises dental and temporo-mandibular joint (TMJ) side effects. Thus, it is configured to minimises or prevent any movement of one or more of the teeth.

### 1.4 Packaging

When developing a new product, a consideration, in addition to the product itself, is the development of a packaging system for the product. Packaging design is helpful to ensure that the products can get to market efficiently and safely.

Several considerations may be considered in the development of a packaging system. The packaging system should be able to adequately protect the product from damage. The packaging system should also allow for the packaged products to shipped easily and economically. Additionally, it may be beneficial for the components of the packaging system be easily and economically manufactured. Furthermore, the packaging system should be designed so that the product can be packaged therein in an efficient manner.

Patient interface systems used for respiratory therapy may also have specific packaging needs. Patient interface systems may need to be packaged sufficiently well to protect easily damaged components. Some components may be damaged by light or moisture and, thus, the packaging system must account for this problem as well.

Prior art packaging systems have failed to combine these considerations into an effective solution to package, protect, and transport patient interface systems.

### 2 BRIEF SUMMARY OF THE TECHNOLOGY

The present technology is directed towards providing medical devices used in the diagnosis, amelioration, treatment, or prevention of respiratory disorders having one or more of improved comfort, cost, efficacy, ease of use and manufacturability.

A first aspect of the present technology relates to apparatus used in the diagnosis, amelioration, treatment or prevention of a respiratory disorder.

Another aspect of the present technology relates to methods used in the diagnosis, amelioration, treatment or prevention of a respiratory disorder.

Another aspect of the present technology is to overcome the shortcomings of the prior art, e.g. to efficiently and economically package patient interface systems with adequate protection.

Another aspect of the present technology is directed to a packaging tray to contain a patient interface system such that the patient interface system is substantially restrained from movement within the packaging tray, e.g., during transport.

Another aspect of the present technology is directed to a packaging tray to contain a patient interface system and spare components for the patient interface system in a compact configuration.

Another aspect of the present technology is directed to a one-piece packaging tray to contain a patient interface system and documentation such as a user guide on opposite sides the packaging tray.

Another aspect of the present technology is directed to a packaging tray for a patient interface system, the patient interface system adapted to provide respiratory therapy. The packaging tray may comprise: at least one cushion assembly region, each shaped to conform to at least a portion of a cushion assembly; a tube region shaped to conform to at least a portion of a tube; a positioning and stabilising structure region shaped to conform to at least a portion of a positioning and structure; and a plurality of flaps adapted to retain documentation for the patient interface system.

In examples (a) each of the plurality of flaps includes a kinked portion, (b) the packaging tray may comprise an outer wall and the plurality of flaps may extend from the outer wall, (c) the outer wall may comprise at least one notch to allow access to the documentation retained by the plurality of flaps, (d) each at least one cushion assembly region may be arranged such that when cushion assemblies are placed in each at least one cushion assembly region, a seal-forming structure of each cushion assembly extends into a plenum chamber of an adjacent cushion assembly, (e) each at least one cushion assembly region may be further arranged such that when cushion assemblies are placed in each at least one cushion assembly region, each cushion assembly does not contact an adjacent cushion assembly, (f) the positioning and stabilising structure region may comprise a pair of rigidiser arm regions and a strap region, (g) each of the pair of rigidiser arm regions may be sloped to conform to a respective rigidiser arm of the patient interface system, (h) the strap region may be shaped and dimensioned to hold straps of a positioning and stabilising structure folded and/or tucked into the strap region, (i) the packaging tray may comprise an inner wall to separate the at least one tube region from the at least one cushion assembly region such that when the tube is placed in the packaging tray, at least a portion of the tube wraps around at least a portion of the inner wall, (j) the packaging tray may comprise a tube surface to support the tube above the positioning and stabilising structure, (k) the packaging tray may comprise a rotatable adapter region shaped to conform to at least a portion of a rotatable adapter, (l) the packaging tray may comprise polypropylene or high-impact polystyrene, and/or (m) the packaging tray may be injection molded.

Another aspect of the present technology is directed to a method for packaging a patient interface system in a packaging tray. The method may comprise: inserting documentation into the packaging tray such that the documentation is retained against the packaging tray with a plurality of flaps of the packaging tray; inserting cushion assemblies into respective cushion assembly regions of the packaging tray; inserting a positioning and stabilising structure into a positioning and stabilising structure region of the packaging tray; and inserting a tube into a tube region of the packaging tray.

In examples (a) each cushion assembly may be inserted into a respective cushion assembly region of the packaging tray such that a seal-forming structure of each cushion assembly extends into a plenum chamber of an adjacent cushion assembly, (b) the method may comprise inserting a rotatable adapter into a rotatable adapter region of the packaging tray, (c) inserting the positioning and stabilising structure into the positioning and stabilising structure region of the packaging tray may comprise inserting rigidiser arms into respective rigidiser arm regions, the rigidiser arm regions shaped and dimensioned to substantially conform to the respective rigidiser arms, (d) inserting the positioning and stabilising structure into the positioning and stabilising structure region of the packaging tray may comprise folding and/or tucking a strap of the positioning and stabilising structure into a strap region of the packaging tray, (e) inserting the tube into the tube region of the packaging tray may comprise at least partially wrapping the tube around an inner wall of the packaging tray and placing at least a portion of the tube on a tube surface of the packaging tray such that the tube is supported above the positioning and stabilising structure, (f) the method may comprise inserting the packaging tray into a box, and/or (g) the packaging tray may comprise polypropylene or high-impact polystyrene and may be injection molded.

Another aspect of the present technology is directed to a packaging tray for a patient interface system adapted to provide respiratory therapy. The packaging tray may comprise: a plurality of cushion assembly regions, each shaped to conform to at least a portion of a cushion assembly; a tube region shaped to conform to at least a portion of a tube; and a positioning and stabilising structure region shaped to conform to at least a portion of a positioning and structure, wherein the cushion assembly regions are shaped and dimensioned to retain and protect respective cushion assemblies in a nested arrangement.

In examples, (a) the packaging tray may comprise a plurality of flaps adapted to retain documentation for the patient interface system and each of the plurality of flaps may include a kinked portion, (b) the packaging tray may comprise an outer wall and the plurality of flaps may extend from the outer wall, (c) the outer wall may comprise at least one notch to allow access to the documentation retained by the plurality of flaps, (d) the cushion assembly regions may be arranged such that when the cushion assemblies are placed in respective cushion assembly regions, a seal-forming structure of one of the cushion assemblies extends into a plenum chamber of an adjacent cushion assembly, (e) the cushion assembly regions may be further arranged such that when cushion assemblies are placed in each cushion assembly region, each cushion assembly does not contact an adjacent cushion assembly, (f) the positioning and stabilising structure region may comprise a pair of rigidiser arm regions and a strap region, (g) each of the pair of rigidiser arm regions may be sloped to conform to a respective rigidiser arm of the patient interface system, (h) the strap region may be shaped and dimensioned to hold straps of a positioning and stabilising structure folded and/or tucked into the strap region, (i) the packaging tray may comprise an inner wall to separate the at least one tube region from the at least one cushion assembly region such that when the tube is placed in the packaging tray, at least a portion of the tube wraps around at least a portion of the inner wall, (j) the packaging tray may comprise a tube surface to support the tube above the positioning and stabilising structure, (k) the packaging tray may comprise a rotatable adapter region shaped to conform to at least a portion of a rotatable adapter, (l) the packaging tray may comprise polypropylene or high-impact polystyrene, (m) the packaging tray may be injection molded, (n) each one of the cushion assembly regions may be shaped and dimensioned to retain and protect a different sized cushion assembly, (o) the cushion assembly regions may be further arranged such that when cushion assemblies are placed in each cushion assembly region, each cushion assembly contacts at least one adjacent cushion assembly, (p) the seal-forming structure may not be substantially deformed by contact with the adjacent cushion assembly, (p) each one of the cushion assembly regions may be a different size, and/or (r) the seal-forming structure of each cushion assembly extends partially into the plenum chamber of the adjacent cushion assembly.

Another aspect of the present technology is directed to a packaging tray for a patient interface system, the patient interface system adapted to provide respiratory therapy. The packaging tray may comprise: at least one cushion assembly region, each shaped to conform to at least a portion of a cushion assembly; a tube region shaped to conform to at least a portion of a tube; a positioning and stabilising structure region shaped to conform to at least a portion of a positioning and structure; and the packaging tray is about 178 mm long, about 116 mm wide, and about 38 mm in height.

In examples (a) the packaging tray may comprise a plurality of flaps adapted to retain documentation for the patient interface system and each of the plurality of flaps includes a kinked portion, (b) the packaging tray may comprise an outer wall and the plurality of flaps may extend from the outer wall, (c) the outer wall may comprise at least one notch to allow access to the documentation retained by the plurality of flaps, (d) each at least one cushion assembly region may be arranged such that when cushion assemblies are placed in each at least one cushion assembly region, a seal-forming structure of each cushion assembly extends into a plenum chamber of an adjacent cushion assembly, (e) each at least one cushion assembly region may be further arranged such that when cushion assemblies are placed in each at least one cushion assembly region, each cushion assembly does not contact an adjacent cushion assembly, (f) the positioning and stabilising structure region may comprise a pair of rigidiser arm regions and a strap region, (g) each of the pair of rigidiser arm regions may be sloped to conform to a respective rigidiser arm of the patient interface system, (h) the strap region may be shaped and dimensioned to hold straps of a positioning and stabilising structure folded and/or tucked into the strap region, (i) the packaging tray may comprise an inner wall to separate the at least one tube region from the at least one cushion assembly region such that when the tube is placed in the packaging tray, at least a portion of the tube wraps around at least a portion of the inner wall, (j) the packaging tray may comprise a tube surface to support the tube above the positioning and stabilising structure, (k) the packaging tray may comprise a rotatable adapter region shaped to conform to at least a portion of a rotatable adapter, (l) the packaging tray may comprise polypropylene or high-impact polystyrene, and/or (m) the packaging tray may be injection molded.

Another aspect of the present technology is directed to a packaging tray for a patient interface system, the patient interface system adapted to provide respiratory therapy. The packaging tray may comprise: at least one cushion assembly region, each shaped to conform to at least a portion of a cushion assembly; a tube region shaped to conform to at least a portion of a tube; a positioning and stabilising structure region shaped to conform to at least a portion of a positioning and structure; and about 89.7% of an area of the packaging tray, when viewed from above, is adapted to retain and protect the patient interface system.

In examples (a) the packaging tray may comprise a plurality of flaps adapted to retain documentation for the patient interface system and each of the plurality of flaps includes a kinked portion, (b) the packaging tray may comprise an outer wall and the plurality of flaps may extend from the outer wall, (c) the outer wall may comprise at least one notch to allow access to the documentation retained by the plurality of flaps, (d) each at least one cushion assembly region may be arranged such that when cushion assemblies are placed in each at least one cushion assembly region, a seal-forming structure of each cushion assembly extends into a plenum chamber of an adjacent cushion assembly, (e) each at least one cushion assembly region may be further arranged such that when cushion assemblies are placed in each at least one cushion assembly region, each cushion assembly does not contact an adjacent cushion assembly, (f) the positioning and stabilising structure region may comprise a pair of rigidiser arm regions and a strap region, (g) each of the pair of rigidiser arm regions may be sloped to conform to a respective rigidiser arm of the patient interface system, (h) the strap region may be shaped and dimensioned to hold straps of a positioning and stabilising structure folded and/or tucked into the strap region, (i) the packaging tray may comprise an inner wall to separate the at least one tube region from the at least one cushion assembly region such that when the tube is placed in the packaging tray, at least a portion of the tube wraps around at least a portion of the inner wall, (j) the packaging tray may comprise a tube surface to support the tube above the positioning and stabilising structure, (k) the packaging tray may comprise a rotatable adapter region shaped to conform to at least a portion of a rotatable adapter, (l) the packaging tray may comprise polypropylene or high-impact polystyrene, and/or (m) the packaging tray may be injection molded.

Another aspect of the present technology is directed to a packaging tray for a patient interface system, the patient interface system adapted to provide respiratory therapy. The packaging tray may comprise: at least one cushion assembly region, each shaped to conform to at least a portion of a cushion assembly; a tube region shaped to conform to at least a portion of a tube; a positioning and stabilising structure region shaped to conform to at least a portion of a positioning and structure; and the packaging tray has a length-width ratio of approximately 1.53, a length-height ratio of approximately 4.68, and a width-height ratio of approximately 3.05.

In examples (a) the packaging tray may comprise a plurality of flaps adapted to retain documentation for the patient interface system and each of the plurality of flaps includes a kinked portion, (b) the packaging tray may comprise an outer wall and the plurality of flaps may extend from the outer wall, (c) the outer wall may comprise at least one notch to allow access tp the documentation retained by the plurality of flaps, (d) each at least one cushion assembly region may be arranged such that when cushion assemblies are placed in each at least one cushion assembly region, a seal-forming structure of each cushion assembly extends into a plenum chamber of an adjacent cushion assembly, (e) each at least one cushion assembly region may be further arranged such that when cushion assemblies are placed in each at least one cushion assembly region, each cushion assembly does not contact an adjacent cushion assembly, (f) the positioning and stabilising structure region may comprise a pair of rigidiser arm regions and a strap region, (g) each of the pair of rigidiser arm regions may be sloped to conform to a respective rigidiser arm of the patient interface system, (h) the strap region may be shaped and dimensioned to hold straps of a positioning and stabilising structure folded and/or tucked into the strap region, (i) the packaging tray may comprise an inner wall to separate the at least one tube region from the at least one cushion assembly region such that when the tube is placed in the packaging tray, at least a portion of the tube wraps around at least a portion of the inner wall, (j) the packaging tray may comprise a tube surface to support the tube above the positioning and stabilising structure, (k) the packaging tray may comprise a rotatable adapter region shaped to conform to at least a portion of a rotatable adapter, (l) the packaging tray may comprise polypropylene or high-impact polystyrene, and/or (m) the packaging tray may be injection molded.

Another aspect of the present technology is directed to a packaging tray for a patient interface system, the patient interface system adapted to provide respiratory therapy. The packaging tray may comprise: three cushion assembly regions, each shaped to conform to at least a portion of a cushion assembly; a tube region shaped to conform to at least a portion of a tube; and a positioning and stabilising structure region shaped to conform to at least a portion of a positioning and structure, wherein the cushion assembly regions are arranged such that each cushion assembly is oriented in a common direction when placed in respective cushion assembly regions.

In examples (a) each of the plurality of flaps includes a kinked portion, (b) the packaging tray may comprise an outer wall and the plurality of flaps may extend from the outer wall, (c) the outer wall may comprise at least one notch to access the documentation retained by the plurality of flaps, (d) each at least one cushion assembly region may be arranged such that when cushion assemblies are placed in each at least one cushion assembly region, a seal-forming structure of each cushion assembly extends into a plenum chamber of an adjacent cushion assembly, (e) each at least one cushion assembly region may be further arranged such that when cushion assemblies are placed in each at least one cushion assembly region, each cushion assembly does not contact an adjacent cushion assembly, (f) the positioning and stabilising structure region may comprise a pair of rigidiser arm regions and a strap region, (g) each of the pair of rigidiser arm regions may be sloped to conform to a respective rigidiser arm of the patient interface system, (h) the strap region may be shaped and dimensioned to hold straps of a positioning and stabilising structure folded and/or tucked into the strap region, (i) the packaging tray may comprise an inner wall to separate the at least one tube region from the at least one cushion assembly region such that when the tube is placed in the packaging tray, at least a portion of the tube wraps around at least a portion of the inner wall, (j) the packaging tray may comprise a tube surface to support the tube above the positioning and stabilising structure, (k) the packaging tray may comprise a rotatable adapter region shaped to conform to at least a portion of a rotatable adapter, (l) the packaging tray may comprise polypropylene or high-impact polystyrene, and/or (m) the packaging tray may be injection molded.

Another aspect of the present technology is directed to a packaging tray for a patient interface system, the patient interface system adapted to provide respiratory therapy. The packaging tray may comprise: at least one cushion assembly region, each shaped to conform to at least a portion of a cushion assembly; a tube region shaped to conform to at least a portion of a tube; a positioning and stabilising structure region shaped to conform to at least a portion of a positioning and structure; and at least about 80% of the volume of the packaging tray is utilized to protect and retain components of the patient interface system.

In examples (a) the packaging tray may comprise a plurality of flaps adapted to retain documentation for the patient interface system and each of the plurality of flaps includes a kinked portion, (b) the packaging tray may comprise an outer wall and the plurality of flaps may extend from the outer wall, (c) the outer wall may comprise at least one notch to allow access to the documentation retained by the plurality of flaps, (d) each at least one cushion assembly region may be arranged such that when cushion assemblies are placed in each at least one cushion assembly region, a seal-forming structure of each cushion assembly extends into a plenum chamber of an adjacent cushion assembly, (e) each at least one cushion assembly region may be further arranged such that when cushion assemblies are placed in each at least one cushion assembly region, each cushion assembly does not contact an adjacent cushion assembly, (f) the positioning and stabilising structure region may comprise a pair of rigidiser arm regions and a strap region, (g) each of the pair of rigidiser arm regions may be sloped to conform to a respective rigidiser arm of the patient interface system, (h) the strap region may be shaped and dimensioned to hold straps of a positioning and stabilising structure folded and/or tucked into the strap region, (i) the packaging tray may comprise an inner wall to separate the at least one tube region from the at least one cushion assembly region such that when the tube is placed in the packaging tray, at least a portion of the tube wraps around at least a portion of the inner wall, (j) the packaging tray may comprise a tube surface to support the tube above the positioning and stabilising structure, (k) the packaging tray may comprise a rotatable adapter region shaped to conform to at least a portion of a rotatable adapter, (l) the packaging tray may comprise polypropylene or high-impact polystyrene, (m) the packaging tray may be injection molded, (n) about 87% to about 96% of the volume of the packaging tray is utilized to protect and retain components of the patient interface system, (o) about 90% to about 93% of the volume of the packaging tray is utilized to protect and retain components of the patient interface system, and/or (p) about 92% of the volume of the packaging tray is utilized to protect and retain components of the patient interface system.

Another aspect of the present technology is directed to a method of managing the production and transportation of a product. The method may comprise manufacturing a first portion of components for a final product deliverable to a consumer at a first location, manufacturing a packaging tray to protect and contain the final product, packaging said first portion of components into said packaging tray, transporting the packaging tray containing said first portion of components to a second location, producing a second portion of components for the final product, packaging said second portion of components into said packaging tray, packaging said packaging tray containing said first portion of components and said second portion of components into a box, said box containing said packaging tray, said first portion of components and said second portion of components comprising the final product and/or transporting said final product to the consumer.

Another aspect of one form of the present technology is a patient interface that is moulded or otherwise constructed with a clearly defined perimeter shape which is intended to match that of an intended wearer.

An aspect of one form of the present technology is a method of manufacturing apparatus.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE SEVERAL VIEWS OF THE DRAWINGS

The present technology is illustrated by way of example, and not by way of limitation, in the figures of the accompanying drawings, in which like reference numerals refer to similar elements including:

### 3.1 Treatment Systems

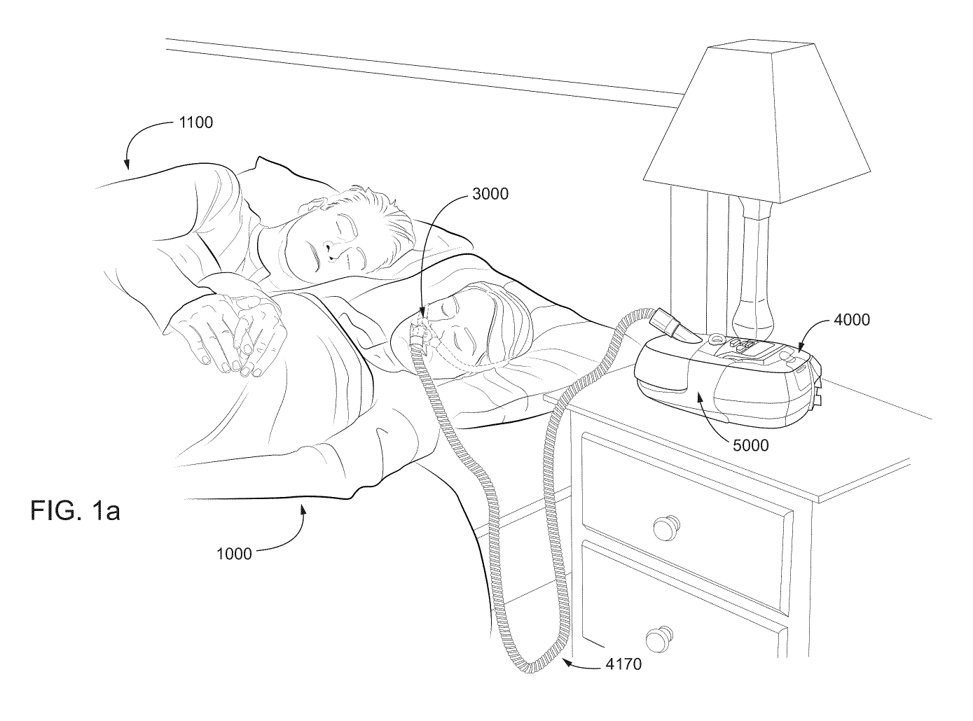

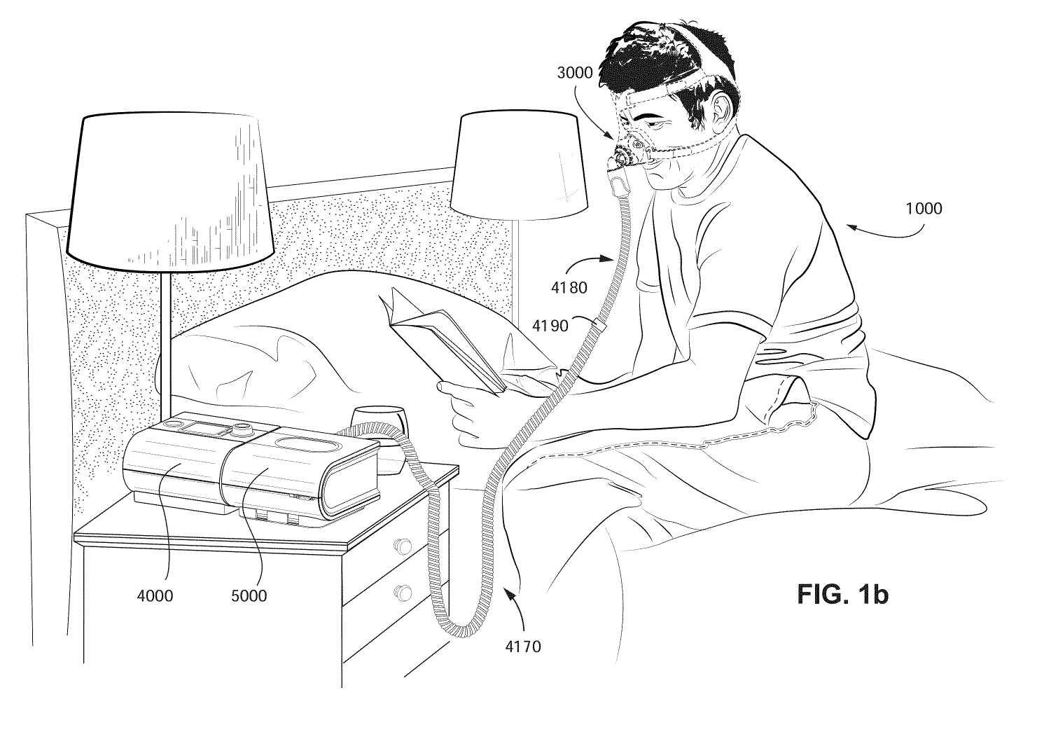

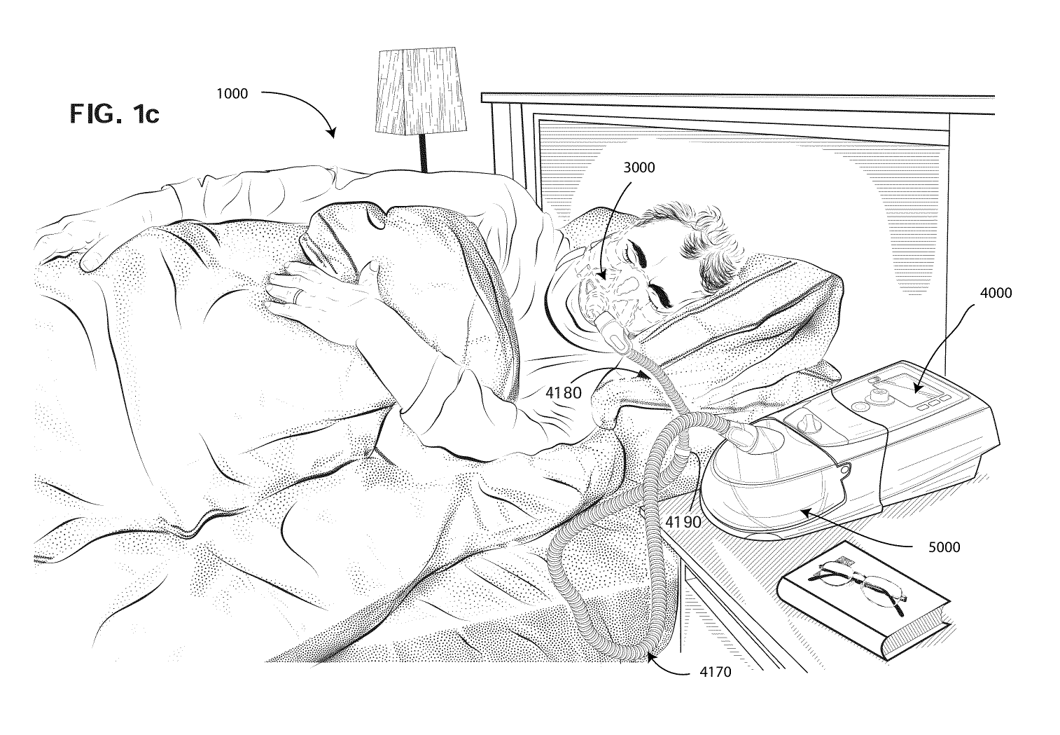

FIG. 1a shows a system in accordance with the present technology. A patient 1000 wearing a patient interface 3000, receives a supply of air at positive pressure from a PAP device 4000. Air from the PAP device 4000 is humidified in a humidifier 5000, and passes along an air circuit 4170 to the patient 1000.

FIG. 1b shows a PAP device 4000 in use on a patient 1000 with a nasal mask.

FIG. 1c shows a PAP device 4000 in use on a patient 1000 with a full-face mask.

### 3.2 Therapy

### 3.2.1 Respiratory System

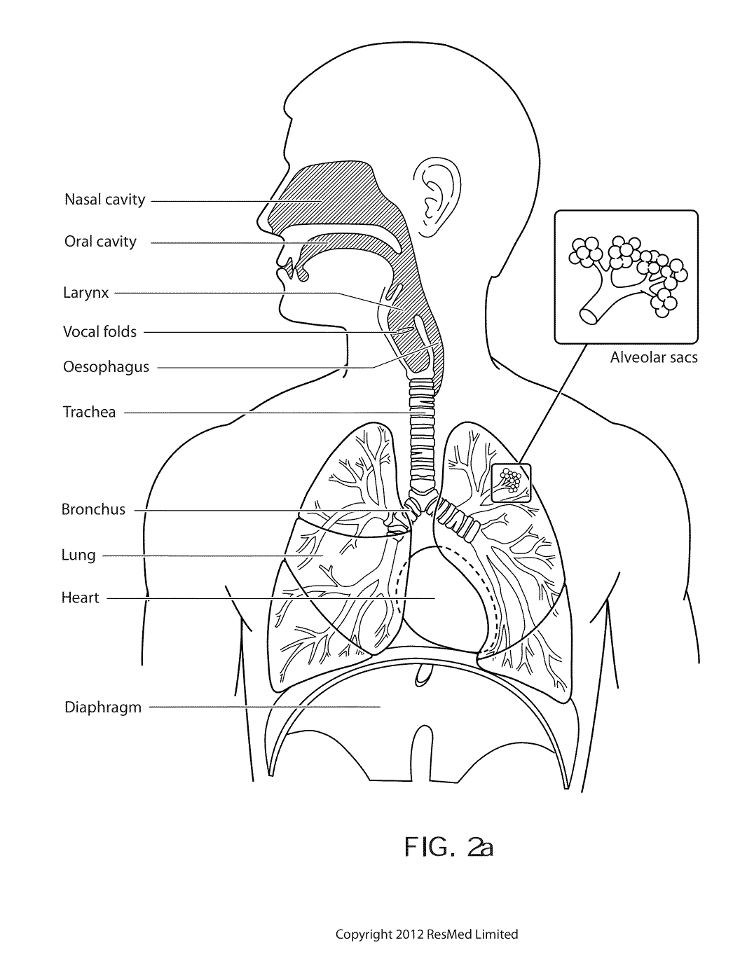

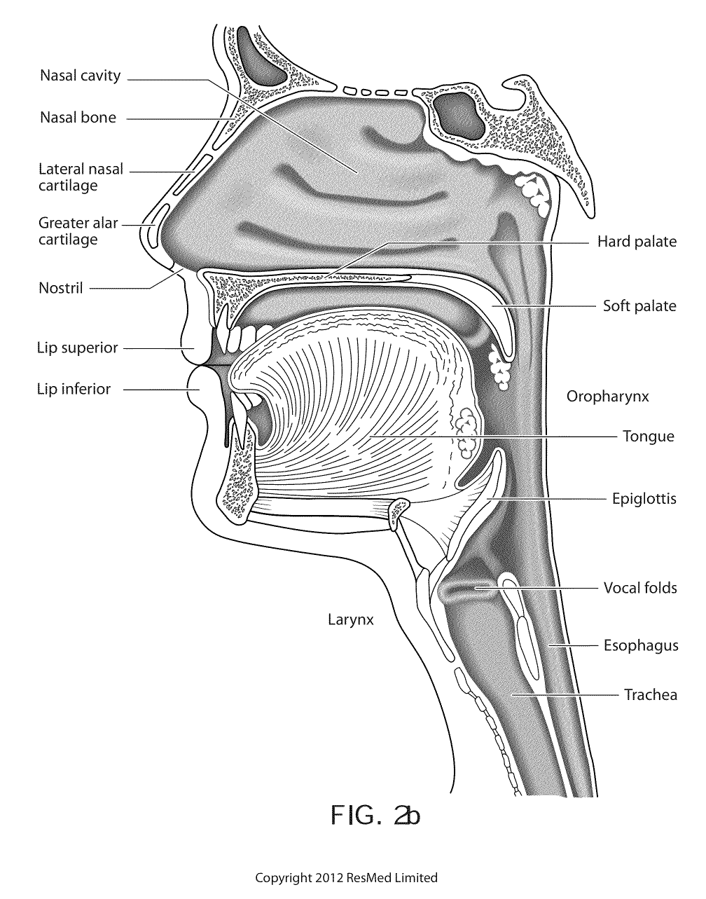

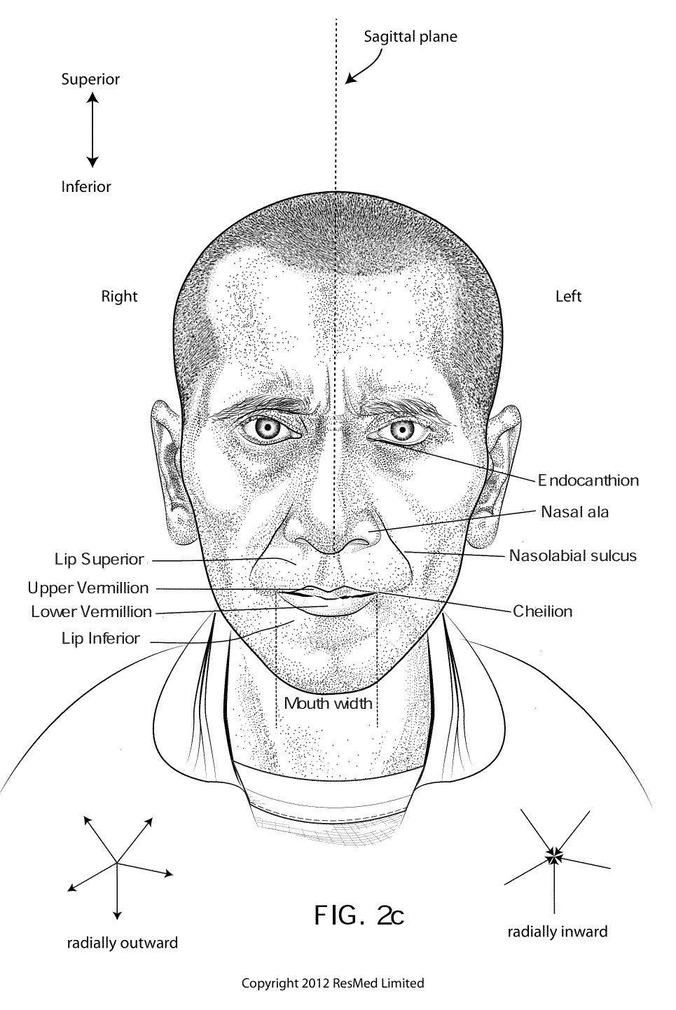

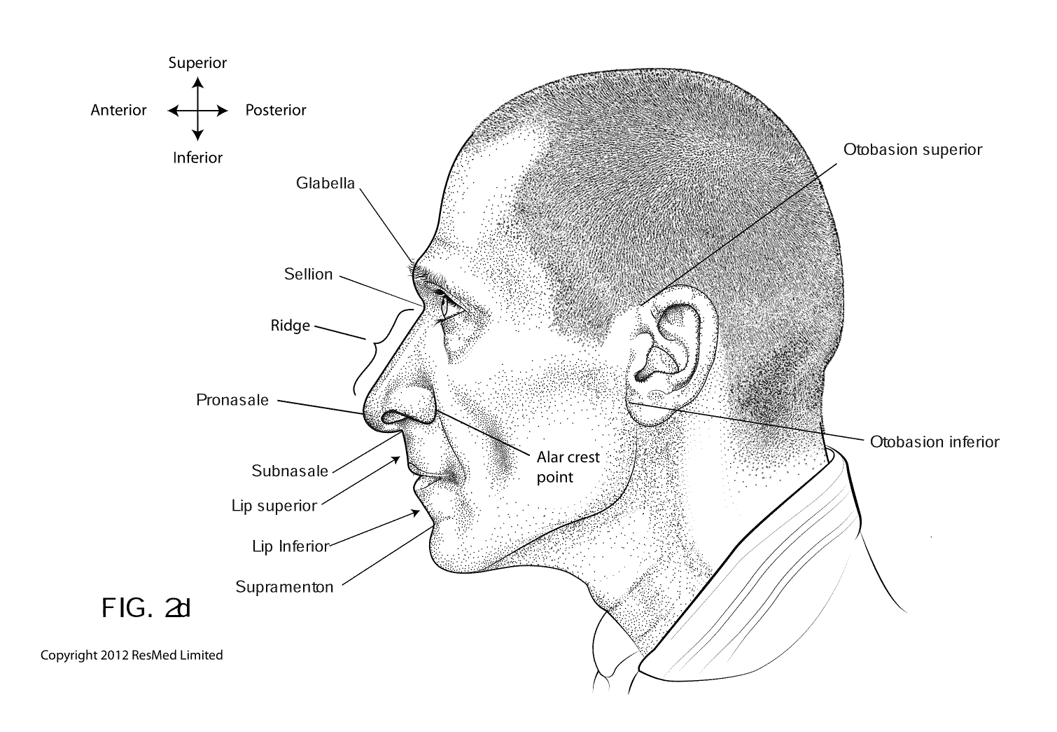

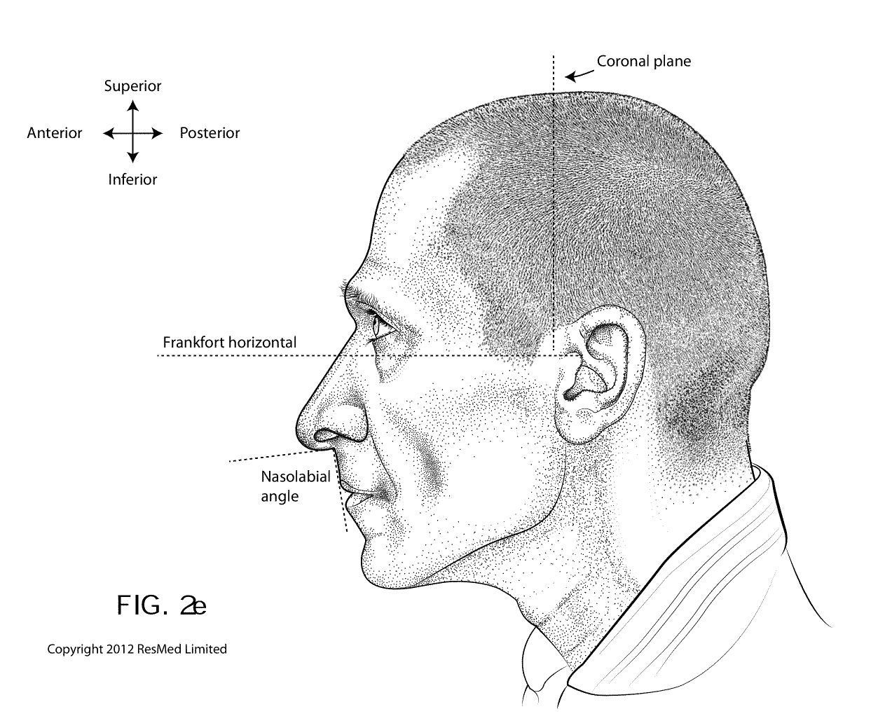

FIG. 2a shows an overview of a human respiratory system including the nasal and oral cavities, the larynx, vocal folds, oesophagus, trachea, bronchus, lung, alveolar sacs, heart and diaphragm.

FIG. 2b shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

### 3.2.2 Facial Anatomy

FIG. 2c is a front view of a face with several features of surface anatomy identified including the lip superior, upper vermillion, lower vermillion, lip inferior, mouth width, endocanthion, a nasal ala, nasolabial sulcus and cheilion.

FIG. 2d is a side view of a head with several features of surface anatomy identified including glabella, sellion, pronasale, subnasale, lip superior, lip inferior, supramenton, nasal ridge, otobasion superior and otobasion inferior. Also indicated are the directions superior & inferior, and anterior & posterior.

FIG. 2e is a further side view of a head. The approximate locations of the Frankfort horizontal and nasolabial angle are indicated.

FIG. 2f shows a base view of a nose.

FIG. 2g shows a side view of the superficial features of a nose.

FIG. 2h shows subcutaneal structures of the nose, including lateral cartilage, septum cartilage, greater alar cartilage, lesser alar cartilage and fibrofatty tissue.

FIG. 2i shows a medial dissection of a nose, approximately several millimeters from a sagittal plane, amongst other things showing the septum cartilage and medial crus of greater alar cartilage.

FIG. 2j shows a front view of the bones of a skull including the frontal, temporal, nasal and zygomatic bones. Nasal concha are indicated, as are the maxilla, mandible and mental protuberance.

FIG. 2k shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter sternocleidomastoid and trapezius.

FIG. 2l shows an anterolateral view of a nose.

### 3.3 PAP Device and Humidifier

FIG. 3a shows an exploded view of a PAP device according to an example of the present technology.

FIG. 3b shows a perspective view of a humidifier in accordance with one form of the present technology.

FIG. 3c shows a schematic diagram of the pneumatic circuit of a PAP device in accordance with one form of the present technology. The directions of upstream and downstream are indicated.

### 3.4 Packaging

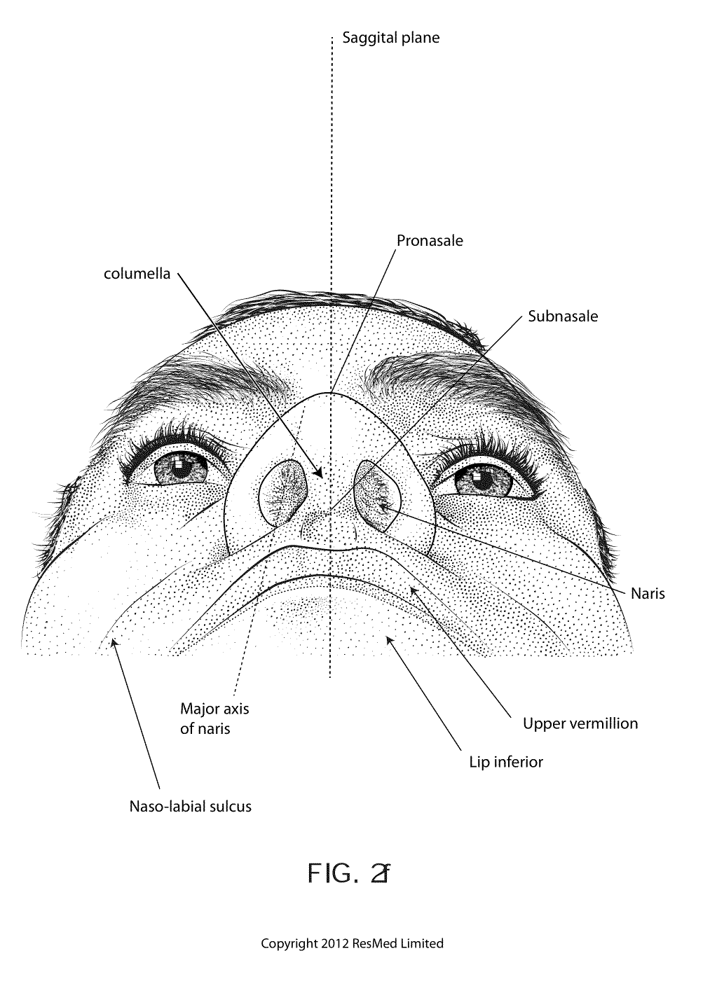

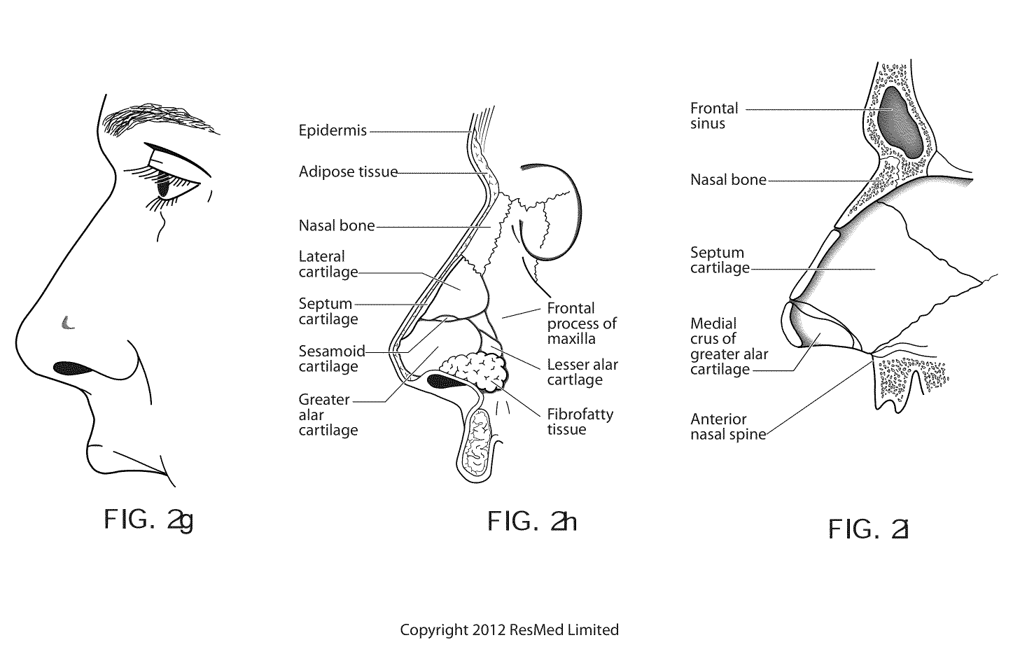

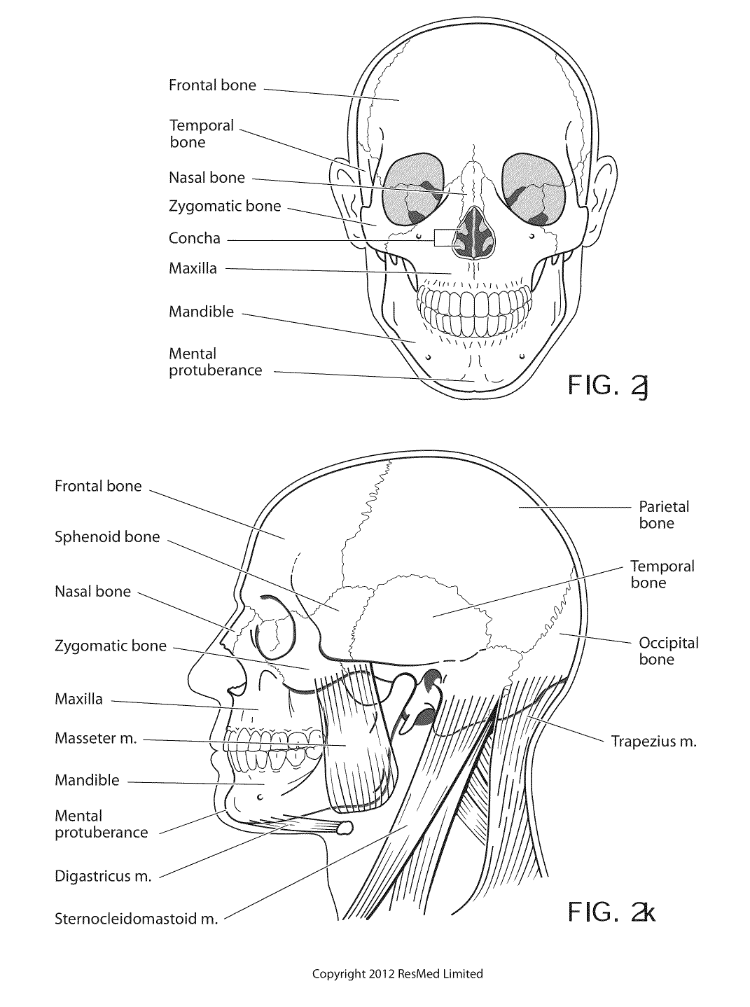

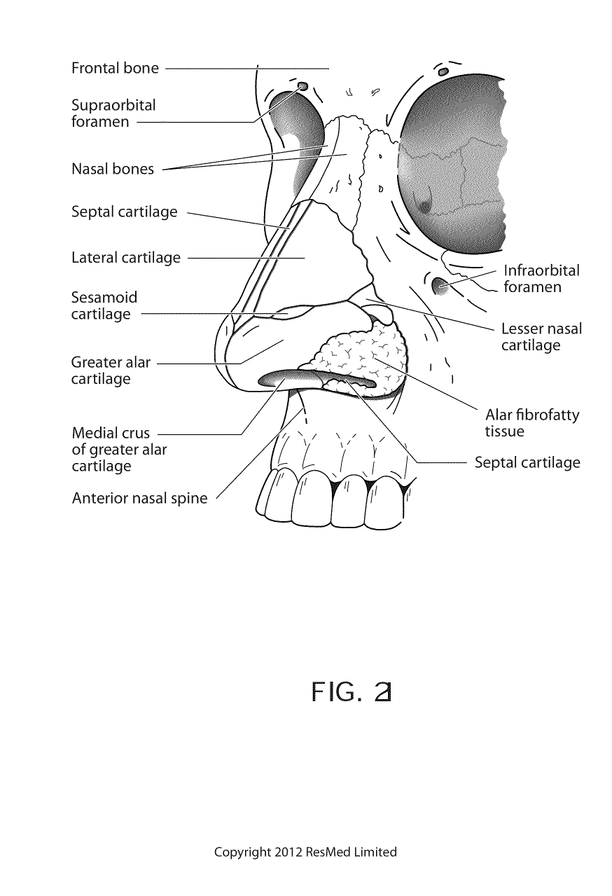

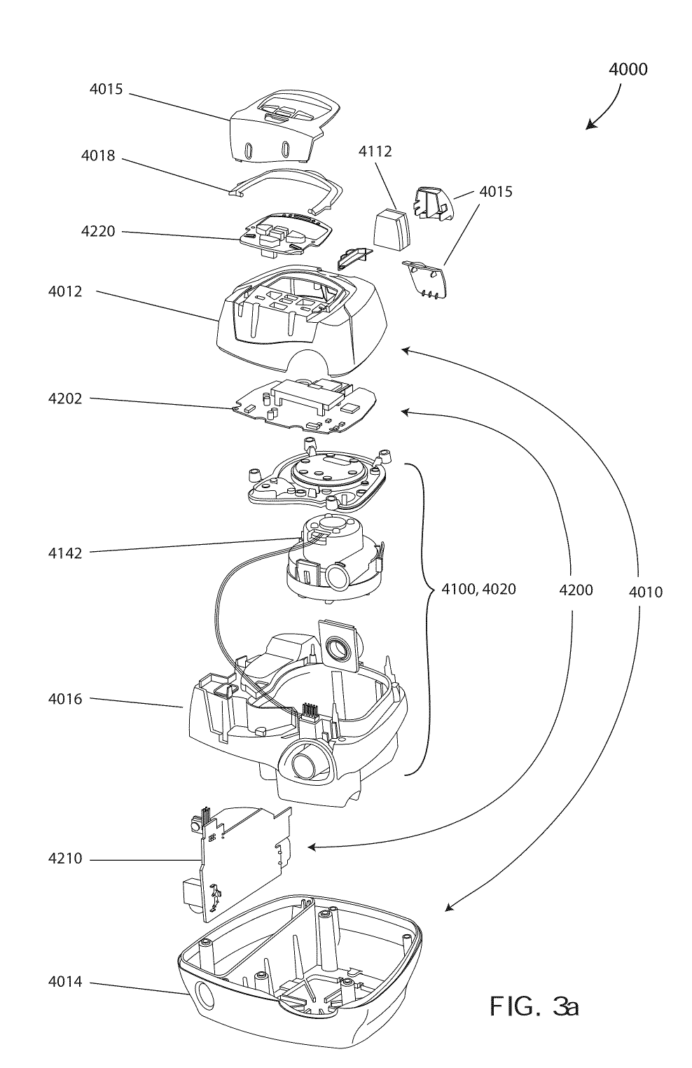

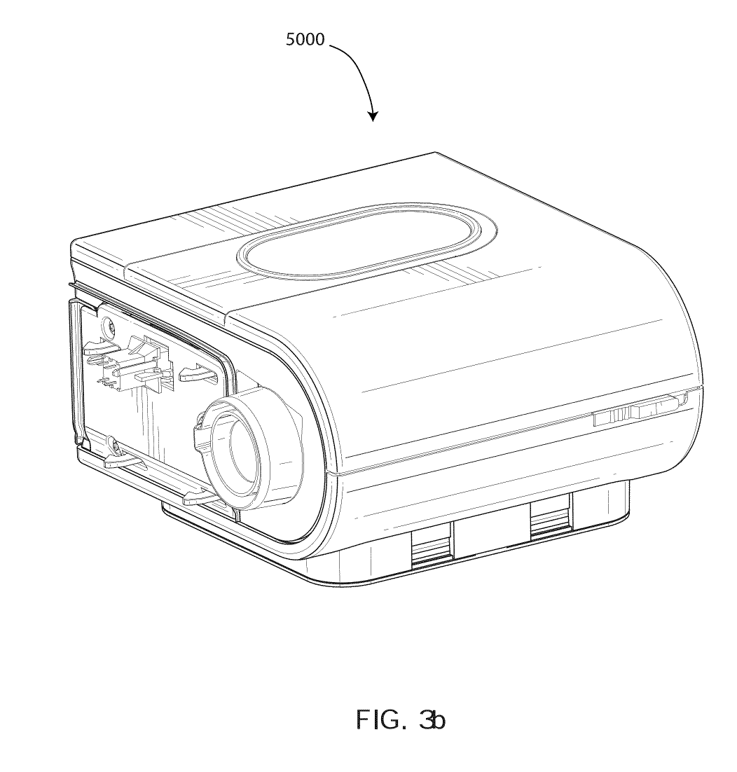

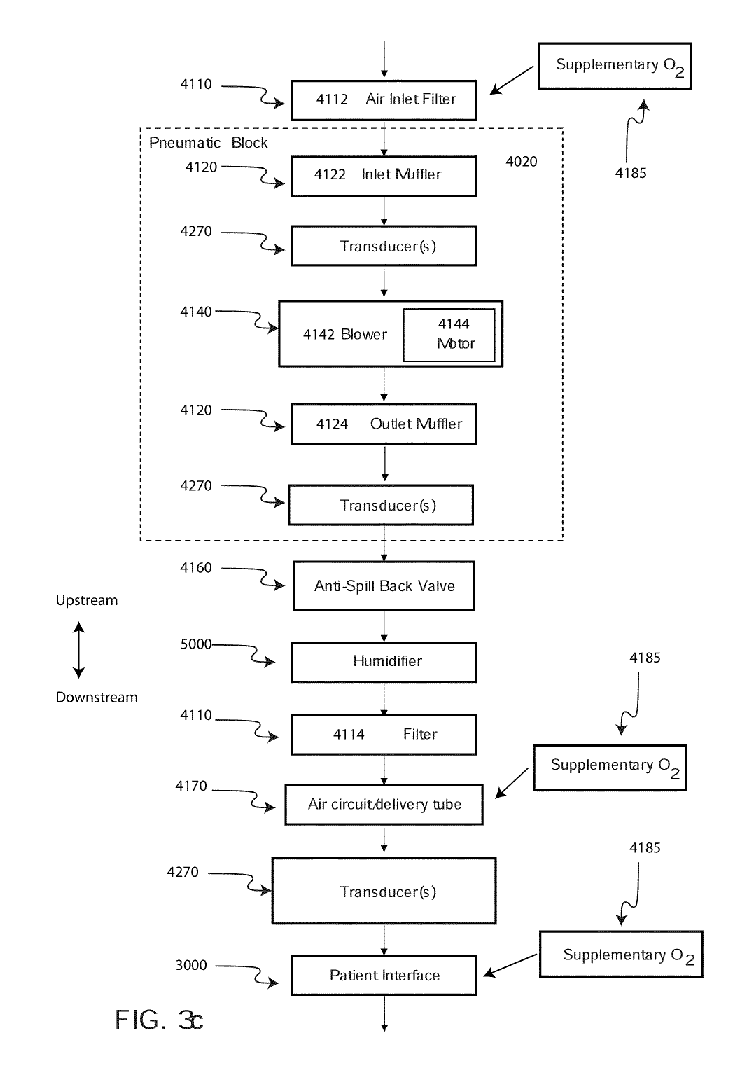

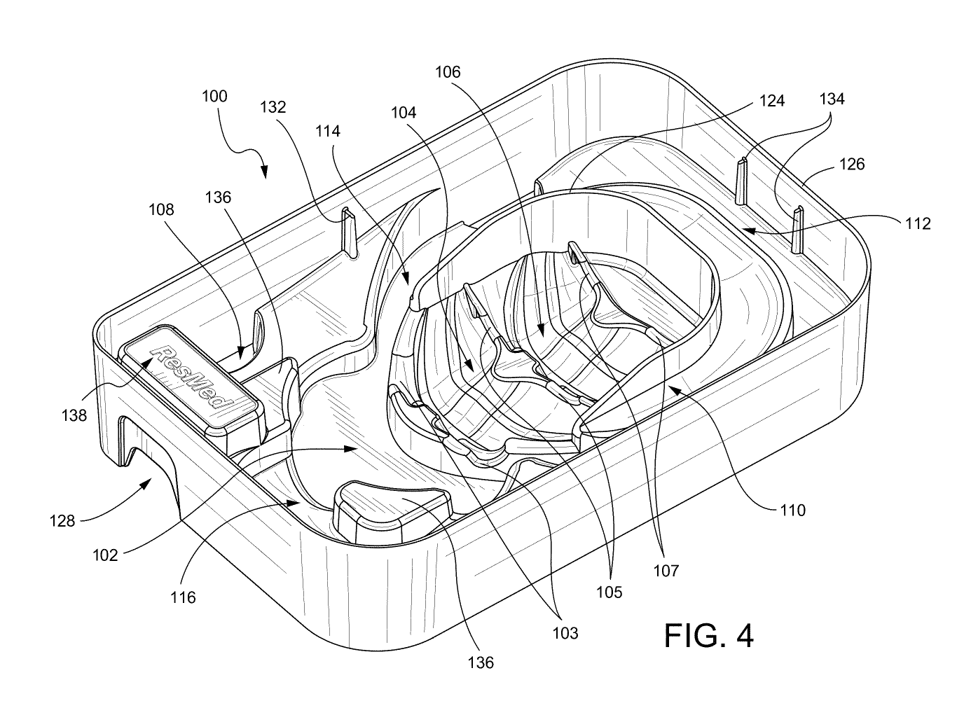

FIG. 4 is a front perspective view of a tray of a packaging system for a patient interface system according to an example of the present technology.

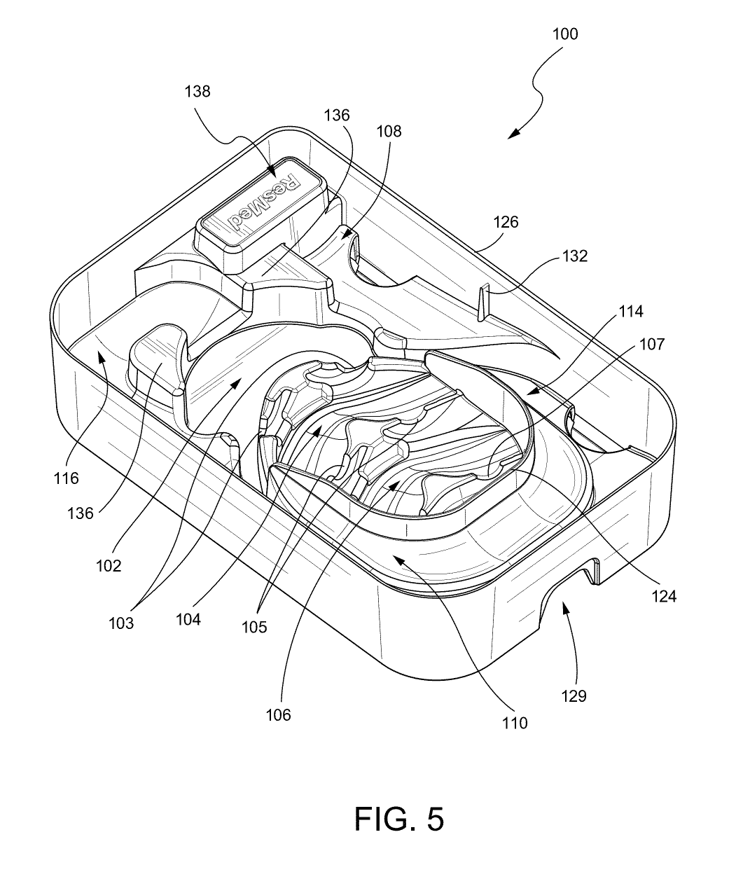

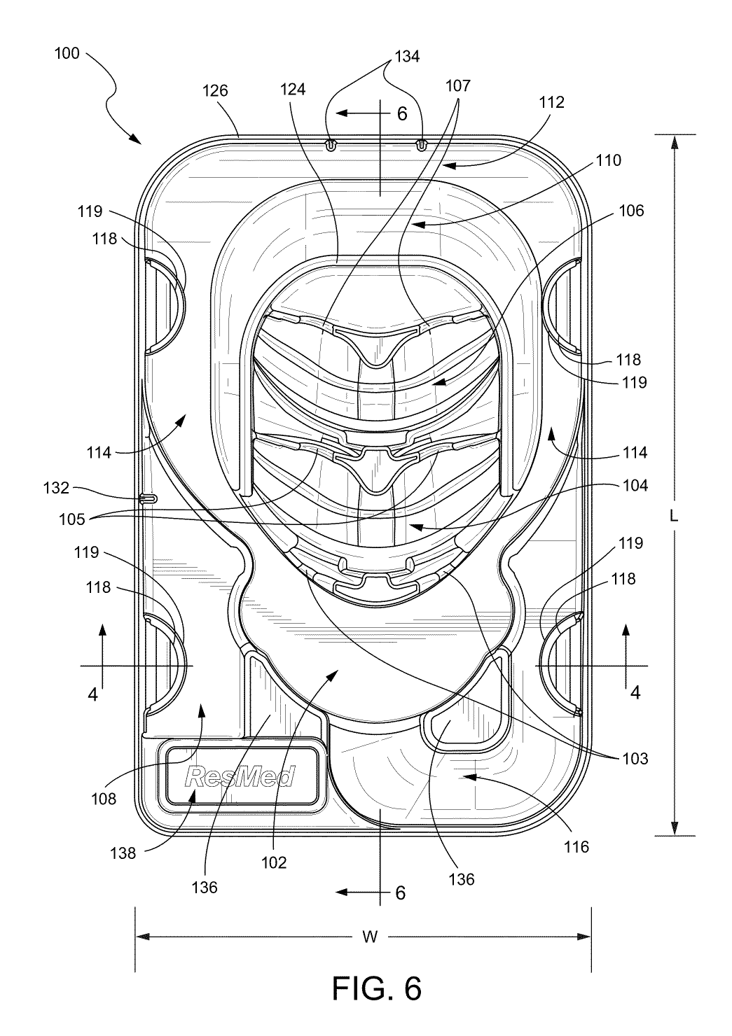

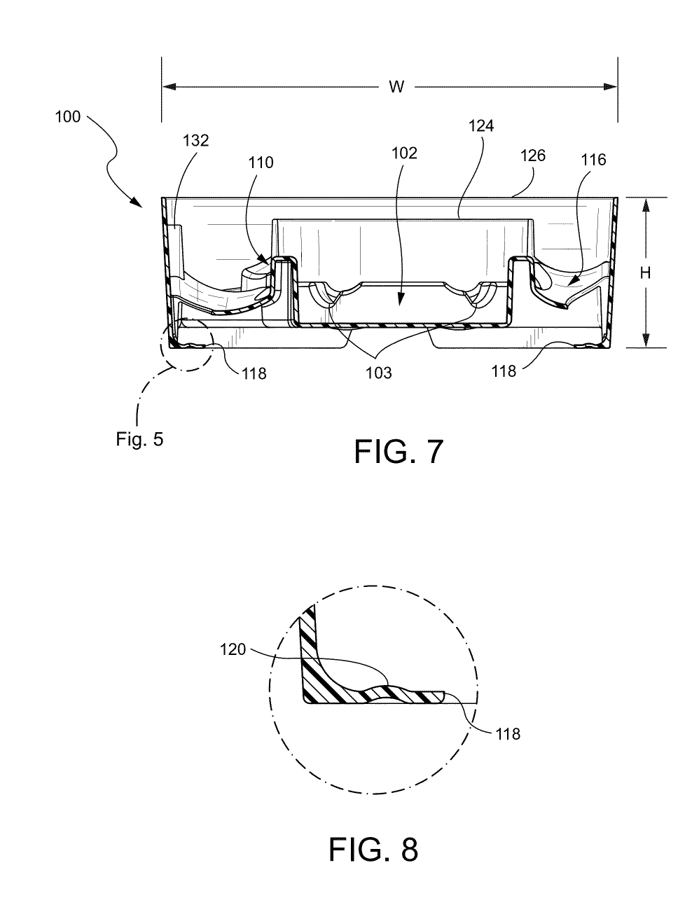

FIG. 5 is a rear perspective view of a tray of a packaging system for a patient interface system according to an example of the present technology.

FIG. 6 is a top view of a tray of a packaging system for a patient interface system according to an example of the present technology.

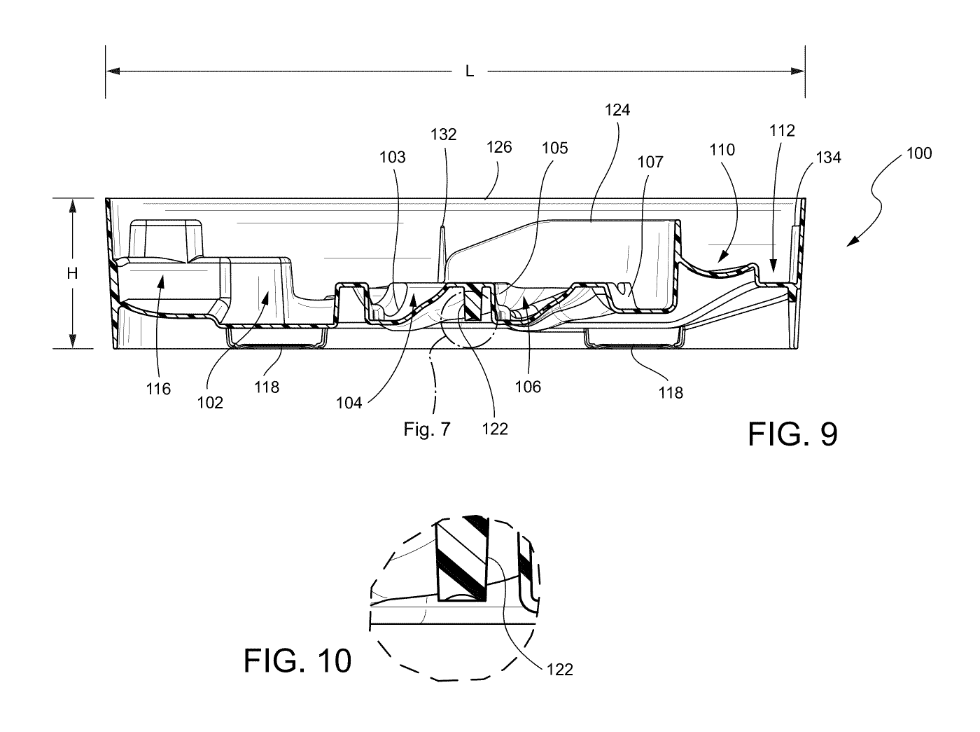

FIG. 7 is a cross-sectional view of a tray of a packaging system for a patient interface system taken through line 4-4 of FIG. 6 according to an example of the present technology.

FIG. 8 is a detailed view of a portion of a tray of a packaging system for a patient interface system as indicated in FIG. 7 according to an example of the present technology.

FIG. 9 is a cross-sectional view of a tray of a packaging system for a patient interface system taken through line 6-6 of FIG. 6 according to an example of the present technology.

FIG. 10 is a detailed view of a portion of a tray of a packaging system for a patient interface system as indicated in FIG. 6 according to an example of the present technology.

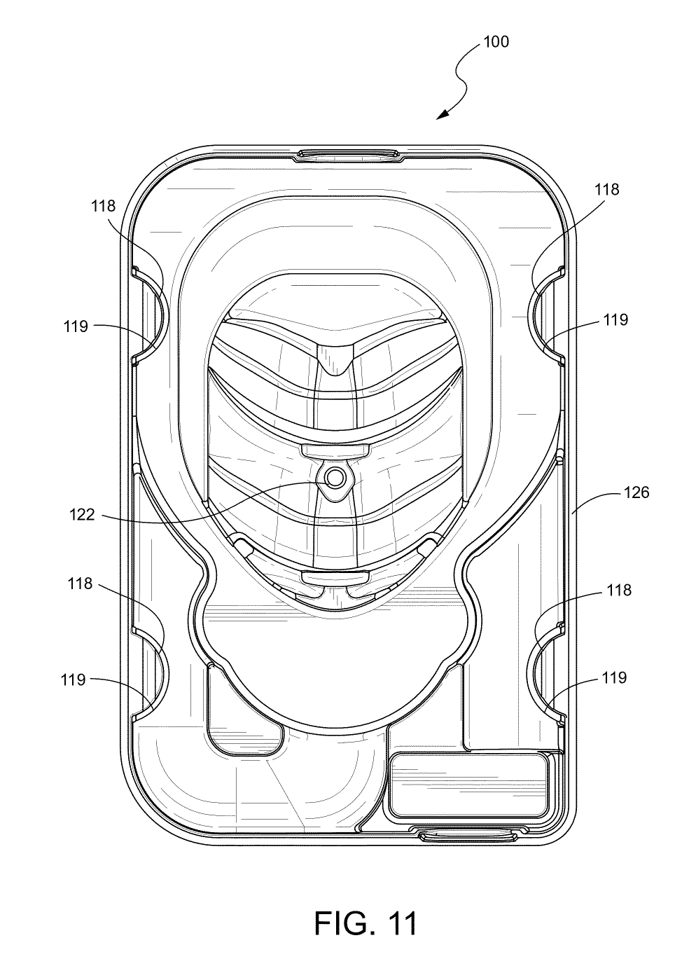

FIG. 11 is a bottom view of a tray of a packaging system for a patient interface system according to an example of the present technology.

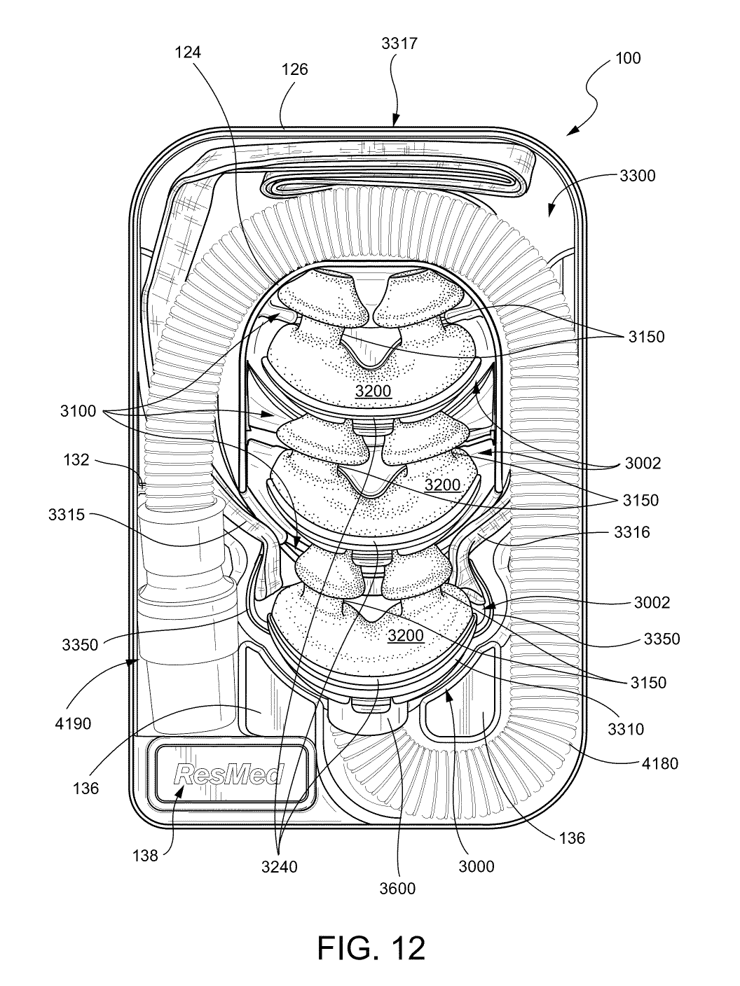

FIG. 12 is a top view of a tray of a packaging system containing a patient interface system according to an example of the present technology.

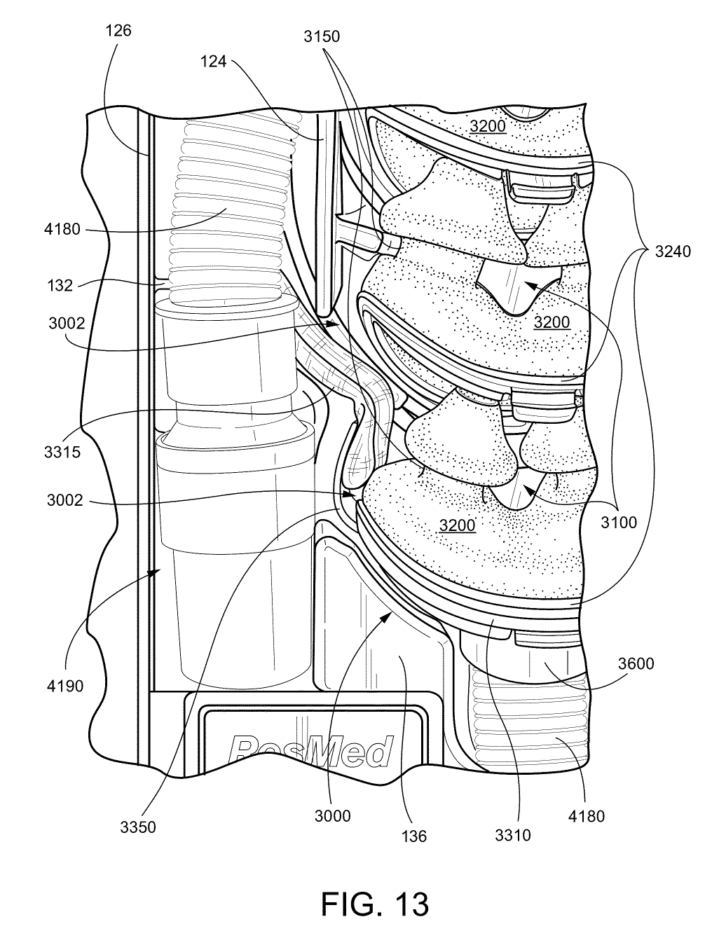

FIG. 13 is a detailed view of a tray of a packaging system containing a patient interface system according to an example of the present technology.

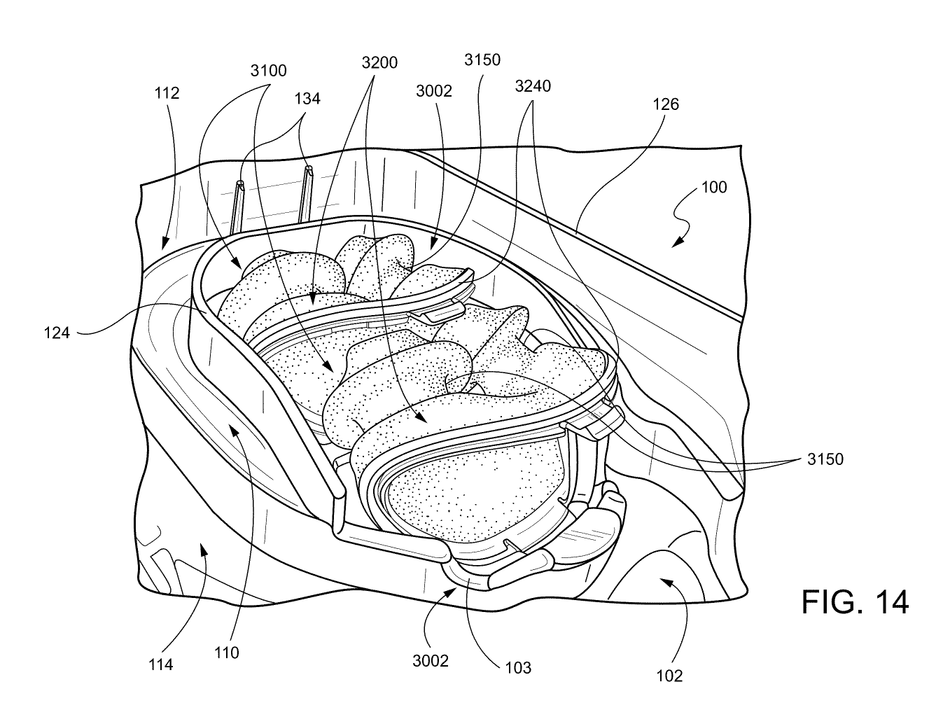

FIG. 14 is a detailed perspective view of a tray of a packaging system containing cushion assemblies of a patient interface system according to an example of the present technology.

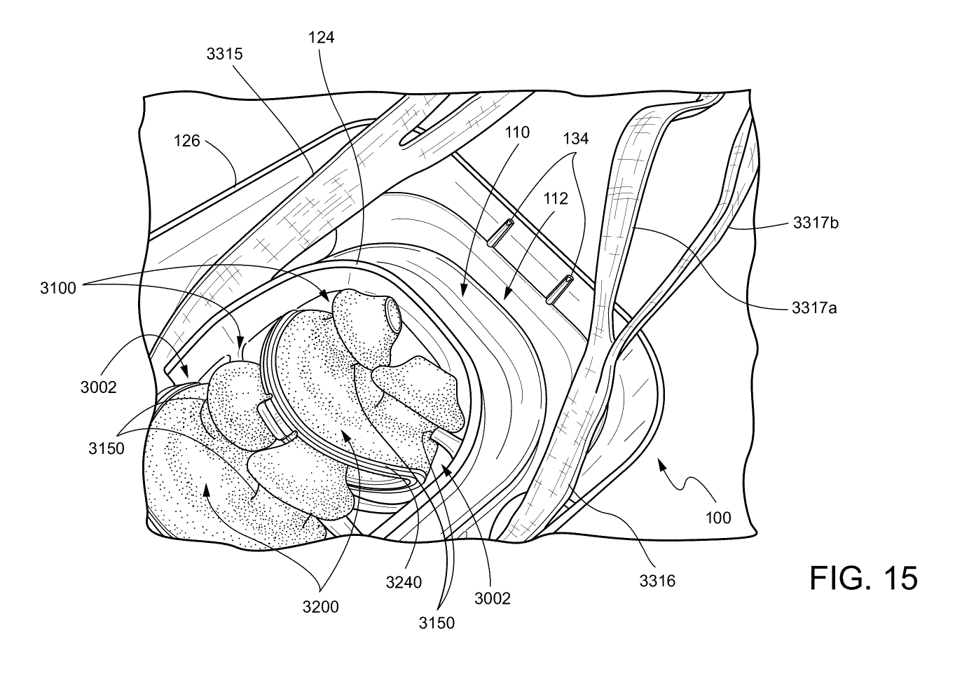

FIG. 15 is a detailed view of a patient interface system being packaged in a tray of a packaging system according to an example of the present technology.

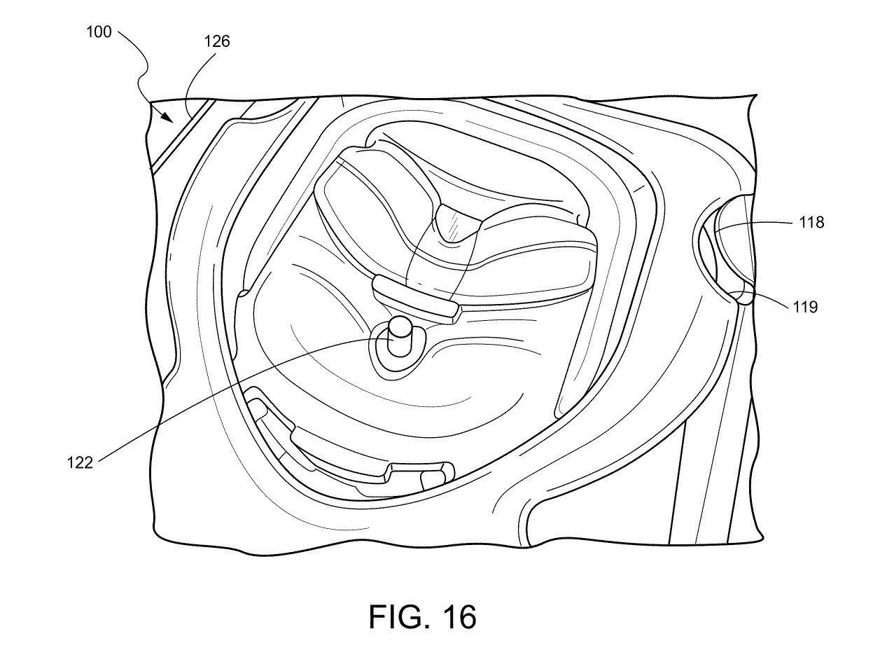

FIG. 16 is a detailed bottom view of a tray of a packaging system containing a patient interface system according to an example of the present technology.

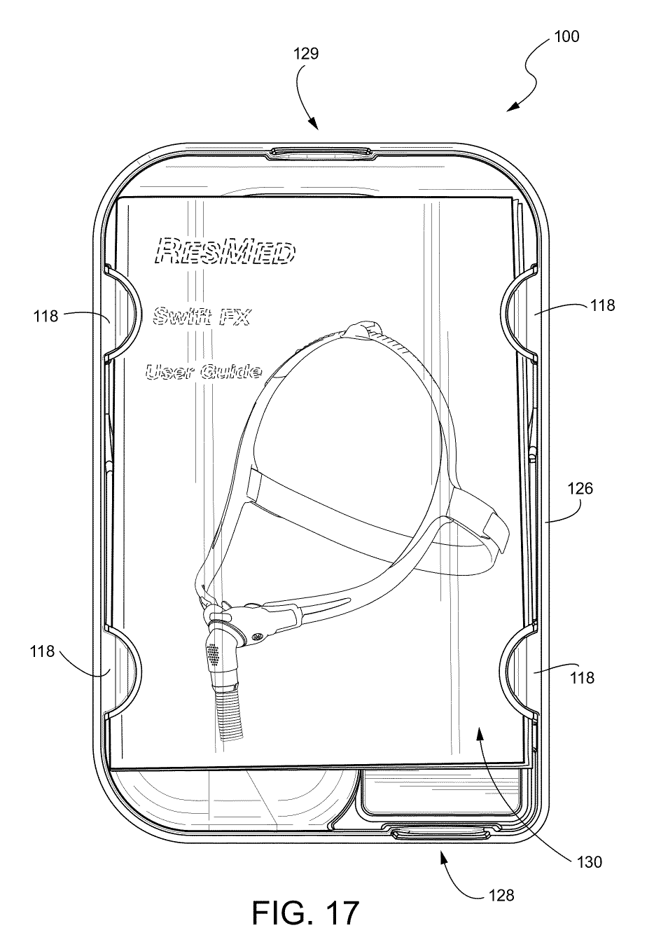

FIG. 17 is a bottom view of a tray of a packaging system for a patient interface system containing a user guide according to an example of the present technology.

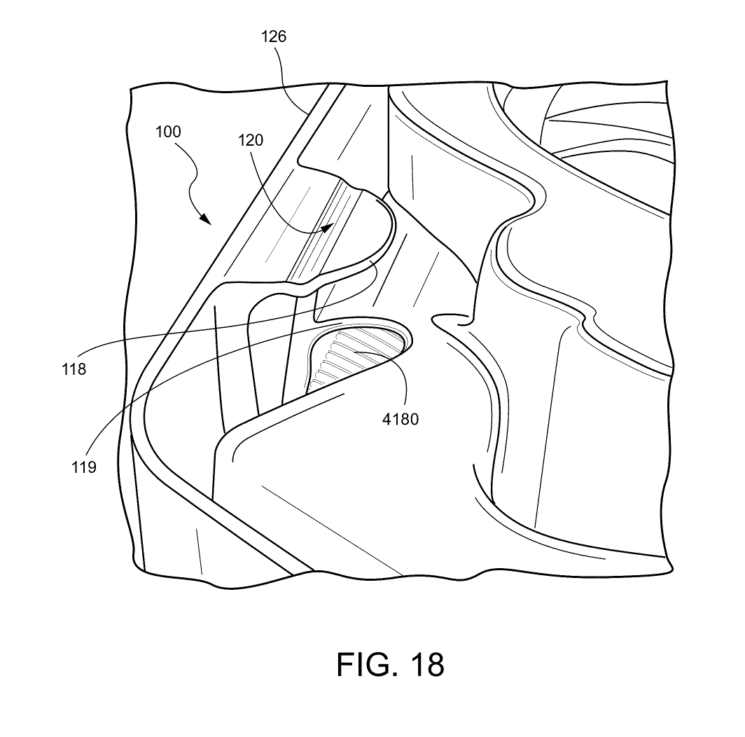

FIG. 18 is a detailed bottom view of a tray of a packaging system for a patient interface system according to an example of the present technology.

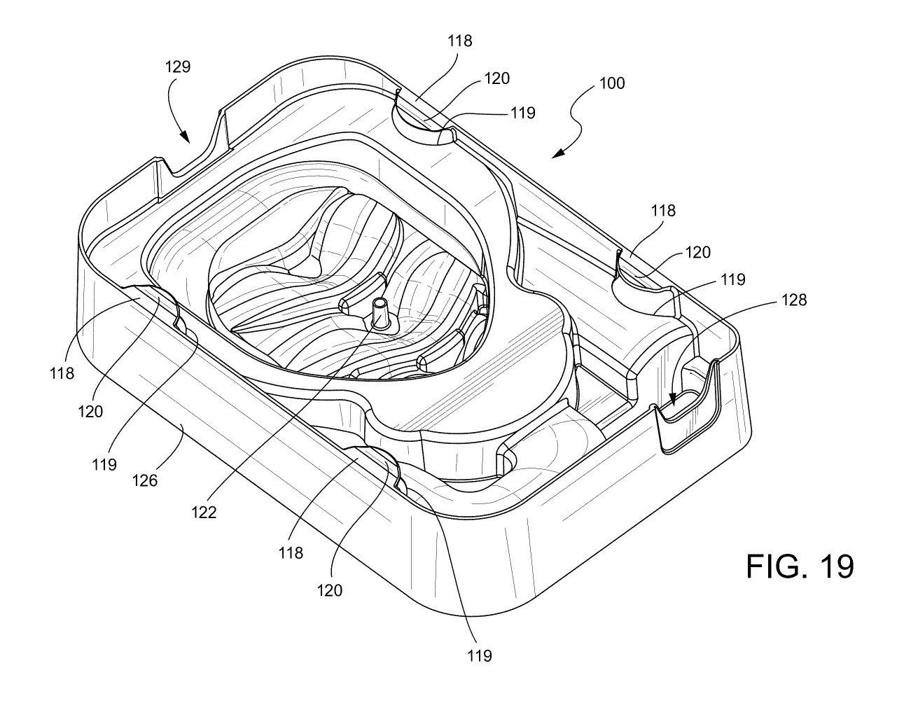

FIG. 19 is a bottom perspective view of a tray of a packaging system for a patient interface system according to an example of the present technology.

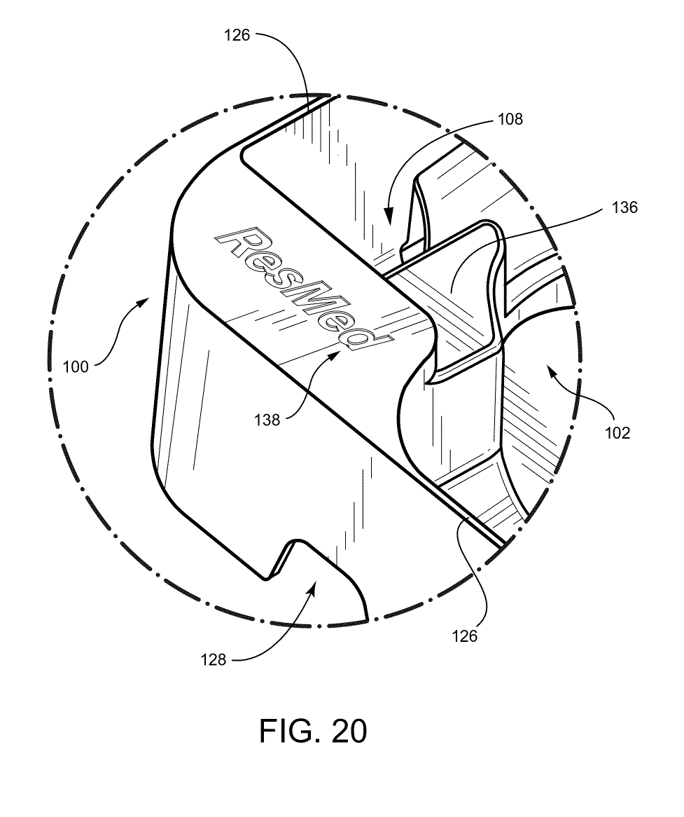

FIG. 20 shows a detailed view of a portion of a tray of a packaging system for a patient interface system according to an example of the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

### 4.1 Treatment Systems

In one form, the present technology comprises apparatus for treating a respiratory disorder. The apparatus may comprise a flow generator or blower for supplying pressurised respiratory gas, such as air, to the patient 1000 via an air delivery tube leading to a patient interface 3000.

### 4.2 Therapy

In one form, the present technology comprises a method for treating a respiratory disorder comprising the step of applying positive pressure to the entrance of the airways of a patient 1000.

### 4.2.1 Nasal CPAP for OSA

In one form, the present technology comprises a method of treating Obstructive Sleep Apnea in a patient by applying nasal continuous positive airway pressure to the patient.

In certain examples of the present technology, a supply of air at positive pressure is provided to the nasal passages of the patient via one or both nares.

In certain examples of the present technology, mouth breathing is limited, restricted or prevented.

### 4.3 Patient Interface

A non-invasive patient interface 3000 in accordance with one aspect of the present technology comprises the following functional aspects: a seal-forming structure 3100, a plenum chamber 3200, a positioning and stabilising structure 3300 and a connection port 3600 for connection to air circuit 4170. In some forms a functional aspect may be provided by one or more physical components. In some forms, one physical component may provide one or more functional aspects. In use the seal-forming structure 3100 is arranged to surround an entrance to the airways of the patient so as to facilitate the supply of air at positive pressure to the airways.

### 4.3.1 Seal-Forming Structure

In one form of the present technology, a seal-forming structure 3100 provides a sealing-forming surface, and may additionally provide a cushioning function.

A seal-forming structure 3100 in accordance with the present technology may be constructed from a soft, flexible, resilient material such as silicone.

In one form, the seal-forming structure 3100 comprises a sealing flange and a support flange. The sealing flange may comprise a relatively thin member with a thickness of less than about 1 mm, for example about 0.25 mm to about 0.45 mm, that extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use. In use the sealing flange can readily respond to system pressure in the plenum chamber 3200 acting on its underside to urge it into tight sealing engagement with the face.

In one form the seal-forming portion of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose; a stalk, a flexible region on the underside of the cone and connecting the cone to the stalk. In addition, the structure to which the nasal pillow of the present technology is connected includes a flexible region adjacent the base of the stalk. The flexible regions can act in concert to facilitate a universal joint structure that is accommodating of relative movement—both displacement and angular—of the frusto-cone and the structure to which the nasal pillow is connected. For example, the frusto-cone may be axially displaced towards the structure to which the stalk is connected.

In one form the non-invasive patient interface 3000 comprises a seal-forming portion that forms a seal in use on an upper lip region (that is, the lip superior) of the patient's face.

In one form the non-invasive patient interface 3000 comprises a seal-forming portion that forms a seal in use on a chin-region of the patient's face.

### 4.3.2 Plenum Chamber

The plenum chamber 3200 may have a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200.

### 4.3.3 Positioning and Stabilising Structure

The seal-forming portion 3100 of the patient interface 3000 of the present technology may be held in sealing position in use by the positioning and stabilising structure 3300.

### 4.3.4 Vent

In one form, the patient interface 3000 includes a vent 3400 constructed and arranged to allow for the washout of exhaled carbon dioxide.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g. a rotatable adapter 4190.

### 4.3.5 Decoupling Structure(s)

In one form the patient interface 3000 includes at least one decoupling structure, for example a rotatable adapter 4190.

### 4.3.6 Connection Port

Connection port 3600 allows for connection to the air circuit 4170.

### 4.3.7 Forehead Support

In one form, the patient interface 3000 includes a forehead support.

### 4.3.8 Anti-Asphyxia

In one form, the patient interface 3000 includes an anti-asphyxia valve.

### 4.3.9 Ports

In one form of the present technology, a patient interface 3000 includes one or more ports that allow access to the volume within the plenum chamber 3200. In one form this allows a clinician to supply supplemental oxygen. In one form this allows for the direct measurement of a property of gases within the plenum chamber 3200, such as the pressure.

### 4.4 Packaging

The packaging system includes an exemplary packaging tray 100 to retain the components of the patient interface system, as shown in FIG. 4. The exemplary packaging tray 100 may be designed to retain a complete patient interface system 3000, in addition to spare parts and documentation for the patient interface system. This may be advantageous in that the process of packaging may be simplified because each and every item to be packaged and delivered will be contained in the packaging tray 100. Moreover, by packaging the patient interface system 3000, along with spare parts and documentation, into a single packaging tray 100, cost may be reduced because fewer pieces are required to contain all of the items to be delivered. Additionally, the packaging tray 100 may be reused at various points from the beginning of production to the end user. For example, various components may be produced separately along the supply chain and the packaging tray 100 may be used to ship the components to another supplier who then adds further components to the packaging tray and so on. This may save costs because only one packaging tray 100 would need to be produced.

The packaging tray 100 may also be formed rigidly. By making the packaging tray 100 rigid, it may be possible to form the walls of the packaging tray more thin so as to waste less space, as will be described in greater detail below.

### 4.4.1 Patient Interface System, Spare Parts, and Documentation

The exemplary packaging system of the present technology may be designed to retain a complete patient interface system 3000, as well as spare parts and documentation for the patient interface system. According to an example of the present technology shown in FIG. 12, the patient interface system 3000 may include cushion assemblies 3002, a short tube 4180, a positioning and stabilising structure 3300, and a rotatable adapter 4190. The patient interface system 3000 may also include a frame 3310 and a connection port 3600 to connect the short tube 4180. Each cushion assembly 3002 may also include a seal-forming structure 3100, a plenum chamber 3200, and a plenum connection region 3240. Additionally, each cushion assembly 3002 may include stalks 3150 connecting the seal-forming structure 3100 to the plenum chamber 3200. The positioning and stabilising structure 3300 may include a back strap 3317, right side strap portion 3315, left side strap portion 3316, and extensions 3350 connecting the frame 3310 to rigidiser arms. The rigidiser arms are not visible because they are contained within the right side strap portion 3315 and the left side strap portion 3316. Exemplary rigidiser arms, as well as other features of exemplary patient interface systems, are disclosed in International Application No. PCT/AU2013/000830, filed Jul. 26, 2013, which is incorporated by reference herein in its entirety.

Spare parts of the patient interface 3000 may also be packaged with the exemplary packaging system. FIG. 12, for example, depicts additional cushion assemblies 3002. The additional cushion assemblies 3002 may be different size cushion assemblies so that the user, when purchasing the packaged complete patient interface system 3000, is provided with different size options to achieve the best fit. This arrangement may be advantageous in that it allows the manufacturer to package and sell uniform individual units capable of meeting different users' needs (e.g. providing various size options to achieve the best fit) and to avoid the increased cost of packaging and selling separate units each designed to fit different users.

As shown in FIG. 17, documentation such as a manual 130 may also be packaged into the packaging system, in addition to the patient interface system 3000 and spare parts. The manual 130 may include instructions for assembly, use, and maintenance of the patient interface system 3000. It may be advantageous to include documentation such as the manual 130 in the same packaging system as the patient interface system 3000 and the spare parts because it allows for compact packaging and the user may be conveniently provided with important information about the product. The manual 130 may also be region specific, for example, such that it is written in a particular language spoken in a particular region.

### 4.4.2 Packaging Tray

A packaging tray 100 in accordance with an example of the present technology, as shown in FIGS. 4-6, may be designed to compactly and securely retain the patient interface system 3000, along with the spare cushion assemblies 3002, and the manual 130. The packaging tray 100 may include several regions shaped to retain the various components of the patient interface system 3000. According to an example of the present technology, these regions may comprise depressions in the top surface of the packaging tray 100.

Cushion assembly regions 102, 104, 106 may be formed on the packaging tray 100 to receive and retain the cushion assemblies 3002. A rotatable adapter region 108 may also be formed on the packaging tray 100, in addition to a rotatable adapter retaining rib 132, to receive and retain the rotatable adapter 4190. A tube surface 110 may also be formed on the packaging tray 100 to support at least a portion of the circumference of the short tube 4180. A strap region 112 may also be formed on the packaging tray 100 to receive and retain at least a portion of the right side strap portion 3315, the left side strap portion 3316, and/or the back strap 3317. Rigidiser arm regions 114 may also be formed on the packaging tray 100 to conform to rigidiser arms of the patient interface system 3000. A tube region 116 may also be formed on the packaging tray 100 to receive and retain at least a portion of the circumference of the short tube 4180. Also, frame retention posts 136 may project from the packaging tray 100 near the tube region 116 and the cushion assembly region 102.

The packaging tray 100 may also include an inner wall 124 to separate the cushion assembly regions 104, 106 from the tube surface 110, the strap region, and the rigidiser arm regions 114. Additionally, the packaging tray 100 may include an outer wall 126 to aid in retaining the patient interface system 3000 and spare cushion assemblies 3002 within the packaging tray. Ribs 134 may also be formed on the outer wall 126 to minimize warp of the packaging tray 100, specifically the outer wall during molding. The ribs 134 may also help to retain the right side strap portion 3315, the left side strap portion 3316, and/or the back strap 3317 in the strap region 112. It should be understood that the inner wall 124 and the outer wall 126 of the packaging tray 100 may also protect its contents (i.e. the patient interface system 3000 and the spare cushion assemblies 3002) from damage. The inner wall 124 and the outer wall 126 may be single walls, according to an example of the present technology. In other words, the inner wall 124 and the outer wall 126 may be a single layer, which may be attributable to injection molding the packaging tray 100, as described further below, as opposed to manufacturing the packaging tray via vacu-forming. The packaging tray 100 also may allow for top loading of the components of the patient interface system 3000 during packaging, thus simplifying the packaging process because all of the components are packaged into one side of the packaging tray. Additionally, it should be understood that the relative position of the regions that contain the components, described in greater detail below, may help to prevent creep and/or material deformation of the components.

Also, the packaging tray 100 may include a support surface 138 having indicia. The indicia on the support surface 138 may be molded into the packaging tray 100 according to an example of the technology. Alternatively, the indicia on the support surface 138 may be embossed or debossed onto the support surface of the packaging tray 100. The indicia on the support surface 138 may be a logo for the product or manufacturer. The indicia on the support surface 138 may also be a trademark or other source identifier. Furthermore, it is envisioned that the indicia on the support surface 138 may provide information such as an indication of orientation of the packaging tray 100 during packaging or for the contents of the packaging tray. The indicia on the support surface 138 may, in another example, provide information such as a size or other product code.

As can be in the Figures, the support surface 138 may be distanced from the outer wall 126. It should also be understood that when the user or packaging staff grips the packaging tray 100 through the notch 128, by extending the index finger therethrough for example, the underside of the support surface 138 may be the boundary of the finger's reach. According to an example of the present technology, the support surface 138 may rise to a height such that it is even with the edge of the inner wall 124 and/or the outer wall 126. According to another example of the present technology, the support surface 138 may be below the height of the edge of the inner wall 124 and/or the outer wall 126.

FIG. 20 shows a support surface 138 according to another example of the present technology. According to this example, the support surface 138 may be formed together with the outer wall 126. Thus, the support surface 138 may rise to a height equal to that of the edge of the outer wall 126. Also, this example shows indicia on the support surface 138.

FIG. 6, which shows a top view of the packaging tray 100 according to an example of the present technology, also shows flaps 118 and cutouts 119. The flaps 118 and cutouts 119 may also be seen in FIG. 11. According to an example of the present technology, the flaps 118 and the cutouts 119 may be curved and generally semi-circular in shape to minimize sharp corners. Fewer sharp corners may be more aesthetically pleasing and may also help to prevent injury to people handling the packaging tray. The flaps 118 may be included to retain the manual 130 as shown in FIG. 14 and discussed in greater detail below. The cutouts 119 may be formed on the packaging tray 100 during and as a result of formation of the flaps 118. Also, the distance between the flaps 118 and the underside of the packaging tray 100 should be optimized to securely retain the manual 130. If the gap between the flaps 118 and the underside of the packaging tray 100 is too large, then the manual could fall out. If the gap between the flaps 118 and the underside of the packaging tray 100 is too small, then it will be difficult to insert and remove the manual 130. Additionally, the flaps 118 may, in part with the outer wall 126, form a base surface upon which the packaging tray 100 may rest.

Also, notches 128, 129, as shown in FIGS. 4 and 5, may be formed on the packaging tray 100 to allow the user to access the manual 130 retained by the flaps 118. Also, the notches 128, 129 may be useful to allow packaging staff to more easily separate individual packaging trays 100 during the packaging process. The notches 128, 129 may allow packaging staff to reach in between stacked packaging trays 100 and pull them apart using, for example, an index finger.

When the packaging tray 100 is packaged with the patient interface system 3000, spare cushion assemblies 3002, and the manual 130 it may then be placed into a box for transport. For example, the packaging tray 100 may be slid into a hollow cardboard box once packaged with the patient interface system 3000, spare cushion assemblies 3002, and the manual 130. The box may be sized and shaped such that the packaging tray 100 and its contents fit snugly therein to prevent damage to the packaging tray and its contents during shipping. The notches 128, 129 may also be beneficially allow the user to remove the packaging tray 100 from the box by providing a grip for the user to grasp and pull the packaging tray from the box with the user's index finger.

The packaging tray 100, according to examples of the present technology, may be formed with rounded corners, as can be seen in the Figures. This may make it easier to slide the packaging tray 100 into the box during packaging. Also, rounded corners may be more aesthetically pleasing. Furthermore, rounded corners may prevent injury to users or packaging staff that may otherwise occur with sharp corners.

According to an example of the technology, the packaging tray 100 may be 178 mm long, 116 mm wide, and 38 mm tall. FIGS. 6, 7, and 9 show length L, width W, and height H dimensions on the exemplary packaging tray 100. The box, not shown but discussed above, may include minimum internal dimensions of 181.5 mm in length, 117.5 mm in width, and 39.5 mm in height. According to another example, the box may have internal dimensions of 182 mm in length, 118 mm in width, and 40 mm in height. In further examples, the preceding dimensions may vary by ±0.8 mm. The box may be hollow and may include a tab with a hole from which the box may be hung.

According to another example of the technology, the packaging tray 100 may be dimensioned according to dimensional ratios. Such an exemplary packaging tray 100 may include a length-width ratio of approximately 1.53, a length-height ratio of approximately 4.68, and a width-height ratio of approximately 3.05.

According to another example, the packaging tray 100 may be shaped and dimensioned to utilize approximately 89.7% of its top surface area for retaining and protecting the components of the patient interface system 3000, when viewed from above as in FIG. 6, for example. In other words, in the packaging tray 100 of this example, approximately 10.3% of the area of the packaging tray 100, when viewed from above, would not comprise the component containing regions discussed in greater detail below. This 10.3% of the area not utilized by the component containing regions may comprise walls, such as the inner wall 124 and the outer wall 126. Thus, with the exemplary dimensions described above, the area of the packaging tray 100, when viewed from above, may be about 20,648 mm² and about 18,521 mm² of that area would be utilized to retain and protect the components of the patient interface system 3000. By designing the packaging tray 100 so efficiently, it may be possible to reduce costs associated with shipping the packaged patient interface system 3000 because shipping costs may be calculated based on shipment volume, rather than weight.

According to further examples of the present technology, the packaging tray 100 may be shaped and dimensioned to utilize at least about 80% of its top surface area for retaining and protecting the components of the patient interface system 3000, when viewed from above as in FIG. 6, for example. According to further examples of the present technology, at least: about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of the top surface area of the packaging tray 100 may be utilized to contain the components.

According to another example of the present technology, the packaging tray 100 may be shaped and dimensioned to efficiently utilize a large percentage of its volume to contain the components of the patient interface system 3000. According to one example, approximately 91% or approximately 92% of the volume of the packaging tray 100 is occupied when packaging is completed. According to another example, approximately 87% to approximately 96% of the volume of the packaging tray 100 may be utilized to contain the components. According to another example, approximately 90% to approximately 93% of the volume of the packaging tray 100 may be utilized to contain the components. According to another example, at least approximately 80% of the volume of the packaging tray 100 may be utilized to contain the components. According to further examples of the present technology, at least: about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of the volume of the packaging tray 100 may be utilized to contain the components.

According to another example of the present technology, the packaging tray 100 may have a total volume of about 769,674 mm³ and it may utilize about 703,980 mm³ to contain the components of the patient interface system. Thus, about 91% of the volume of the packaging tray 100 may be utilized.

According to another example of the present technology, the packaging tray 100 may have a total volume of about 769,674 mm³ and it may utilize about 711,858 mm³ to contain the components of the patient interface system. Thus, about 92% of the volume of the packaging tray 100 may be utilized.

### 4.4.2.1 Cushion Assembly Regions

As discussed above, the packaging tray 100 may include a plurality of cushion assembly regions 102, 104, 106, as shown in FIGS. 4-6, that each receives and retains a cushion assembly 3002. FIG. 11 shows a cross-sectional view of the packaging tray 100 to depict the profile of the cushion assembly regions 102, 104, 106. FIGS. 12-15 show how the cushion assemblies 3002 may be inserted to respective ones of the cushion assembly regions 102, 104, 106.

FIGS. 4-6 and 9 show the shape and contour of the unoccupied cushion assembly regions 102, 104, 106. Each of the cushion assembly regions 102, 104, 106 may be shaped to substantially conform to the shape of the cushion assemblies 3002 that are placed therein. Such an arrangement may help to minimize concentrated stresses on the cushion assemblies 3002. Additionally, as discussed above, each cushion assembly 3002 may be of a different size and, thus, each of the cushion assembly regions 102, 104, 106 may be sized differently to better conform to the cushion assembly that is to be placed therein. The cushion assembly regions 102, 104, 106 may also be shaped to at least partially conform to the plenum connection regions 3240 that are part of the cushion assemblies 3002. Additionally, the cushion assembly regions 102, 104, 106 may be formed asymmetrically on the packaging tray 100 relative to a bisecting longitudinal axis of the packaging tray. This may allow for more efficient use of space on the packaging tray 100, ultimately leading to a more compact and efficient packaging arrangement.

The cushion assembly regions 102, 104, 106 may be formed and/or arranged on the packaging tray 100 so that each cushion assembly 3002 is oriented in the same direction when placed into its respective cushion assembly region. FIGS. 12-15 depict how the cushion assemblies 3002 may be arranged in the same orientation such that the seal-forming structure 3100 of one cushion assembly may be adjacent to the plenum chamber 3200. This may also allow for a nesting arrangement wherein the seal-forming structure 3100 of one cushion assembly 3002 may be located at least partially within the plenum chamber 3200 of the adjacent cushion assembly. This may allow for a compact packaging arrangement by utilizing a portion of the vacant space within the plenum chamber 3200 by occupying it, at least partially, with the adjacent seal-forming structure 3100. In further examples, the cushion assembly regions 102, 104, 106 may be arranged such that adjacent cushion assemblies 3002 contact one another or such that adjacent cushion assemblies do not contact one another. In examples where adjacent cushion assemblies 3002 contact one another, the cushion assembly regions 102, 104, 106 may be arranged such that the seal-forming structure 3100 of one cushion assembly is not compressed or deformed by contact with the plenum connection region 3240 of the adjacent cushion assembly

The packaging tray 100 may also include stalk regions 103, 105, 107, shown in FIGS. 4-7 and 9, where respective stalks 3150 of the seal-forming structures 3100 of the cushion assemblies 3002 may fit. The stalk regions 103, 105, 107 may be curved to conform to the shape of the stalks 3150. The stalks 3150 of the seal-forming structures 3100 may fit into the respective stalk regions 103, 105, 107 when packaging the cushion assemblies 3002 and the stalk regions may help to retain the cushion assemblies in the nested arrangement discussed above. Additionally, the stalk regions 103, 105, 107 may also help to prevent the adjacent cushion assemblies 3002 from contacting one another by retaining them in their respective cushion assembly regions 102, 104, 106.

The cushion assembly region 102 may also be shaped to at least partially conform to and retain the frame 3310 of the patient interface 3000. As can be seen in FIGS. 12 and 13, the cushion assembly region 102 may contain the frame 3310 connected to one of the cushion assemblies 3002. By assembling one of the cushion assemblies 3002 with the frame 3310, a more compact packaging arrangement may be achieved. Additionally, by supporting the cushion assemblies 3002 as shown, creep and/or permanent deformation of the cushion assemblies may be minimized because the cushion assemblies are adequately supported in a substantially unstressed state and may remain so during potentially extended periods of time in the packaging tray 100.

Near the cushion assembly region 102, frame retention posts 136 may also project from the packaging tray 100. The frame retention posts 136 may provide additional protection and retention for the frame 3310 and vents (not visible in these views) located on the frame on both sides of the connection port 3600, as can be seen in FIGS. 12 and 13. The frame retention posts 136 may prevent rubbing of the vents and/or may protect the vents from damage from other components or the packaging tray 100 while in the packaging tray to ensure that the vents are maintained in high-quality, undamaged condition when the user receives the patient interface system 3000. While the vents are not visible, it should be understood that when the patient interface system is packaged into the packaging tray 100, the vents will be located adjacent to respective frame retention posts 136.

### 4.4.2.2 Flaps, Cutouts, and Notches

As discussed above, it may be advantageous to design the packaging tray 100 to be able to carry documentation, such as the manual 130, to make the packaging system more compact. The packaging tray 100 may be formed with features such as the flaps 118, shown in FIGS. 6-9, 11, 17, and 19, to retain the manual 130. The flaps 118 may retain the manual 130 in the packaging tray 100, as shown in FIG. 17, by holding the manual against the underside of the features that retain the patient interface 3000 and the spare cushion assemblies 3002. Thus, the flaps 118 may deflect to allow the manual 130 to fit in the underside of the packaging tray 100 and the flaps may be in tension to help urge the manual against the underside of the packaging tray. The manual 130 may be retained to the underside of the packaging tray 100 with a friction fit by the flaps 118.

While the Figures show that in the disclosed examples of the packaging tray 100 there are four flaps 118, it should be understood that two flaps may be sufficient to retain the manual 130 or it may be necessary to provide more than four flaps.

FIG. 8 also shows kinks 120 that may be formed on the flaps 118. The kinks 120 may be formed on the flaps 118 to help the flaps 118 maintain their shape and avoid warping due to shrinkage during the process of molding the packaging tray 100. As the packaging tray 100 cools following molding it may shrink and the sides of the packaging tray may deform slightly. However, it may be advantageous to avoid having the flaps 118 shrink such that they are angled toward the underside of the packaging tray 100 so that the manual 130 cannot be placed under the flaps and against the underside of the packaging tray. Also, the kinks 120, by projecting toward the packaging tray 100 from the flaps 118, may help to retain the manual 130 by providing a further surface for retention.

Also, forming the flaps 118 on the packaging tray 100 may result in the cutouts 119, shown in FIGS. 6, 11, 16, 18, and 19, being formed. The cutouts 119 may be the part of the packaging tray 100 that is removed of the material that forms the flaps 118. In other words, during molding, the cutouts 119 are formed in the packaging tray 100 as the flaps 118 are cut away.

As can be seen in FIG. 17, the manual 130 occupies substantially all of the bottom surface area of the packaging tray 100. Sizing the pages of the manual 130 to be nearly equal to the bottom surface area of the packaging tray 100 may be advantageous in that fewer pages are necessary for the manual, allowing it to be thinner while containing a given amount of information.

According to another example of the present technology, when the manual 130 is placed into the packaging tray 100, the cutouts 119 may allow packaging staff to see the manual held on the underside of the packaging tray while viewing the packaging tray from above. Thus, the cutouts 119 may allow packaging staff an easy way to ensure that the manual 130 has been packaged without having to turn over the packaging tray 100. This may help to avoid shipping the packaged patient interface system 3000 without the manual 130.

As discussed above, documentation such as the manual 130 may be retained against the underside of the packaging tray 100 with a friction fit by the flaps 118. Thus, it may also be advantageous to provide the user with a simple way to access and remove the manual 130. The notches 128, 129 shown in FIGS. 4, 5, 18, and 19 may allow the user access to a side of the manual 130 to remove it from the packaging tray 100. It should also be understood that during the packaging process, the notches 128, 129 may also allow the packaging staff to more easily insert the manual 130 into the packaging tray 100. Additionally, the notches 128, 129 may allow packaging staff to more easily separate stacked packaging trays 100 during the packaging process. Furthermore, the notch 128 provided near the rotatable adapter region 108 may be offset to the side of the packaging tray 100. By locating the notch 128 to the side, packaging staff may be able to reach deeper in between stacked packaging trays 100 with the index finger, thus making it easier to separate individual packaging trays. The notches 128, 129 may also help the user remove the packaging tray 100 from a box (not shown) that contains the packaged packaging tray. Thus, the user may open an end of the box that contains the packaging tray 100 and remove the packaged packaging tray therefrom by inserting the index finger, gripping, and pulling the packaging tray at one of the notches 128, 129.

As can be seen in FIGS. 4, 5, and 19 notches 128, 129 are provided at two sides of the packaging tray 100. It should be understood that only one notch may be provided on one side of the packaging tray 100 or more than two notches may also be provided on one or more sides of the packaging tray.

### 4.4.2.3 Strap Region and Rigidiser Arm Regions

The packaging tray 100 may also include the strap region 112 and the rigidiser arm regions 114 to receive and retain at least portions of the positioning and stabilising structure 3300. FIGS. 4-6 and 11 show views of the strap region 112 and the rigidiser arm regions 114.

The rigidiser arms of the positioning and stabilising structure 3300 shown in cross-referenced International Application No. PCT/AU2013/000830 may be curved in three dimensions, as disclosed therein, to fit the contours of the face of a user. To better support and retain the rigidiser arms of the positioning and stabilising structure 3300 it may be advantageous to shape the rigidiser arm regions 114 to at least partially conform to the curvature of the rigidiser arms. By so doing the packaging tray 100 may be able to better support the rigidiser arms so that they are not placed under unnecessary stress while in the packaging tray. Moreover, by providing the rigidiser arm regions 114 in the packaging tray 100, creep of the rigidiser arms can be reduced by supporting the rigidiser arms in a position such that the rigidiser arms are as unstressed as possible. Thus, the sloped profile of the rigidiser arm regions 114 and the split arrangement of the rigidiser arm regions, such that the rigidiser arms are splayed widely, allows the rigidiser arms to be supported with minimal stress to reduce creep while packaged.

FIGS. 12, 13, and 15 show views of the patient interface system 3000 packaged in the packaging tray 100. While the rigidiser arms are not visible in these views because they are inside of the right side strap portion 3315 and the left side strap portion 3316 these strap portions can be partially seen extending across the rigidiser arm regions 114 from the extensions 3350.

The strap region 112 may be provided to the packaging tray 100 to receive and retain strap portions of the positioning and stabilising structure 3300. FIG. 15 shows the strap region 112 with the right side strap portion 3315, the left side strap portion 3316, and the back strap 3317 not occupying the strap region. This view may represent an intermediary step in the packaging process. In FIG. 12 the strap region 112 can be seen occupied at least partially by the right side strap portion 3315, the left side strap portion 3316, and the back strap 3317. The strap region 112 may be shaped and sized such that the right side strap portion 3315, the left side strap portion 3316, and the back strap 3317 may be easily tucked into the strap region during the packaging process.

### 4.4.2.4 Tube Region, Tube Surface, and Rotatable Adapter Region

The packaging tray 100 may also include the tube region 116 shown in FIGS. 4-7 and 9. The tube region 116 may be shaped and sized to receive and retain at least a portion of the short tube 4180. The tube region 116 may also be curved to direct the short tube 4180 around the perimeter of the packaging tray 100. FIG. 12 shows a view of the short tube 4180 occupying the tube region 116. By wrapping the short tube 4180 around the perimeter of the packaging tray 100 this may allow for a more compact packaging of the patient interface system 3000 because it may follow substantially all of the longitudinal sides of the packaging tray 100. According to one example of the present technology, the short tube 4180 may follow approximately the entire perimeter of the packaging tray 100 when packaged. For example, FIG. 12 shows that all but a corner of the perimeter of the packaging tray 100 is occupied by the short tube 4180. While the short tube 4180 is designed to be flexible, it should be understood that the curves of the tube region 116 should not be so sharp as to put concentrated strain on the short tube, which may cause permanent material deformation, as it sits in the packaging tray. Also, by having rounded corners on the packaging tray 100 this may provide a truss-like support for the short tube 4180 to spread the load of its weight. In turn this may help to reduce damage to the short tube 4180 that may otherwise occur if the corners were sharp. The tube region 116 may also have a varying arc to minimize creep of the short tube 4180 and/or to prevent it from setting.

Also, it should be understood that the tube region 116 may be slightly raised up from the cushion assembly region 102. The short tube 4180 originates from the frame 3310 at the connection port 3600 and raising the tube region allows the short tube to be supported with minimal stress on the joint between the short tube and the connection port because the short tube does not hang down from the connection port. Also, the tube region 116 may have a profile that is at least partially curved to better conform to and retain at least a portion of the circumference of the short tube 4180.

The tube region 116 may also be raised from the cushion assembly region 102 to provide a more completely complementary recess in which the cushion assembly 3002 and the frame 3310 are retained. By forming the cushion assembly region 102 to more completely surround the cushion assembly 3002 and the frame 3310, these components, in addition to the vents on the frame, may be better protected. Additionally, this arrangement may protect the frame 3310 and the vents from the surface of the cushion assembly region 102.

FIGS. 4-7, 9, 14, and 15 also show the tube surface 110 that may be formed on the packaging tray 100. The tube surface 110 may be provided on the packaging tray 100 to support the short tube 4180 as it wraps around the packaging tray 100 in substantially a complete loop, as shown in FIG. 12. The tube surface 110 may also be provided to support the short tube 4180 above the rigidiser arms and the right side strap portion 3315, the left side strap portion 3316, and the back strap 3317. By raising the short tube 4180 above the rigidiser arms and the strap portions, the short tube is out of the way of the rigidiser arms as they are supported on the packaging tray 100 by the rigidiser arm regions 114 such that they are not substantially deformed. This is advantageous because the short tube 4180 is designed to be flexible so curving it up and around in the packaging tray 100 does not cause significant stress to the short tube. Also, the inner wall 124 may be formed on the packaging tray 100 to prevent the short tube 4180 and the cushion assemblies from contacting one another. The inner wall 124 may provide additional support for the short tube 4180 as well.

Furthermore, the tube region 116 and the tube surface 110 may help to minimize creep of the short tube 4180 once it is packaged in the packaging tray 100. By supporting the short tube 4180 in an unstressed position creep of the short tube can be reduced.

The packaging tray 100 may also include the rotatable adapter region 108 shown in FIGS. 4-7. The rotatable adapter region 108 may be shaped and sized to at least partially conform to the rotatable adapter 4190. The rotatable adapter retaining rib 132 may also be provided to help secure the rotatable adapter 4190 in the rotatable adapter region 108. In one example, the rotatable adapter 4190 may be retained in the rotatable adapter region 108 by abutting against the rotatable adapter retaining rib 132. As the short tube 4180 is wrapped around the packaging tray 100, the rotatable adapter 4190 may be retained in the rotatable adapter region 108 to prevent it from contacting the other components of the patient interface system 3000. Also, the rotatable adapter 4190 is located at an end of the short tube 4180 opposite the frame 3310 and by retaining each end of the short tube in separate regions of the packaging tray 100, the short tube itself may also be more securely retained.

### 4.4.2.5 Manufacturing the Packaging Tray

According to an example of the present technology, the packaging tray 100 may be produced by an injection molding process. A further example of the packaging tray 100 may be formed from a hot tip injection method that may result in a hot tip gate 122, shown in FIGS. 9-11, 16, and 18, formed on the packaging tray. The exemplary packaging tray 100 may be formed from polypropylene or high-impact polystyrene. Also, it should be understood that injection molding the packaging tray 100 may allow it be more rigid relative to a tray that was vacu-formed, for example. Injection molding the packaging tray 100 may also allow the packaging tray to be formed with several different surface finishes. Thus, the various component containing regions may have different surface finishes that are selected based on the particular component held in the particular region. Also, injection molding allows the packaging tray 100 to be made in any of an unlimited range of colors.

Furthermore, injection molding may allow for easily forming various indicia into the packaging tray 100. For example, the indicia of the support surface 138 may be formed by injection molding. It is also envisioned that indicia may be molded on other parts of the packaging tray 100 as well. For example, the various component containing regions may include indicia molded thereon to indicate which component belongs in that particular region. Indicia may also be molded to indicate orientation of components when they are placed into their respective regions. Other identifying information such as sizes, model numbers/names, part numbers/names, etc., may be provided as indicia molded onto the packaging tray 100.

During the formation of the packaging tray 100, as the packaging tray cools, it may shrink in the mold tool and this may result in warping as discussed above. Features such as the kinks 120 discussed above may be included on the packaging tray 100 to account for warping resulting from the shrinkage.

### 4.5 PAP Device

A PAP device 4000 in accordance with one aspect of the present technology comprises mechanical and pneumatic components 4100, electrical components 4200 and is programmed to execute one or more algorithms. The PAP device may have an external housing 4010, formed in two parts, an upper portion 4012 of the external housing 4010, and a lower portion 4014 of the external housing 4010. In alternative forms, the external housing 4010 may include one or more panel(s) 4015. The PAP device 4000 may comprise a chassis 4016 that supports one or more internal components of the PAP device 4000. In one form a pneumatic block 4020 is supported by, or formed as part of the chassis 4016. The PAP device 4000 may include a handle 4018.

The pneumatic path of the PAP device 4000 may comprise an inlet air filter 4112, an inlet muffler, a controllable pressure device capable of supplying air at positive pressure (e.g., a controllable blower 4142), and an outlet muffler. One or more pressure sensors and flow sensors may be included in the pneumatic path.

The pneumatic block 4020 may comprise a portion of the pneumatic path that is located within the external housing 4010.

The PAP device 4000 may have an electrical power supply 4210 and one or more input devices 4220. Electrical components 4200 may be mounted on a single Printed Circuit Board Assembly (PCBA) 4202. In an alternative form, the PAP device 4000 may include more than one PCBA 4202.

### 4.5.1 PAP Device Mechanical & Pneumatic Components

### 4.5.1.1 Air Filter(s)

A PAP device 4000 in accordance with one form of the present technology may include an air filter 4110, or a plurality of air filters 4110.

In one form, an inlet air filter 4112 is located at the beginning of the pneumatic path upstream of a controllable blower 4142. See FIG. 3c.

In one form, an outlet air filter 4114, for example an antibacterial filter, is located between an outlet of the pneumatic block 4020 and a patient interface 3000. See FIG. 3c.

### 4.5.1.2 Pressure Device

In a form of the present technology, a pressure device for producing a flow of air at positive pressure is a controllable blower 4142. For example the blower 4142 may include a brushless DC motor with one or more impellers housed in a volute. The blower 4142 may be capable of delivering a supply of air, for example about 120 litres/minute, at a positive pressure in a range from about 4 cmH2O to about 20 cmH2O, or in other forms up to about 30 cmH2O.

### 4.6 Humidifier

### 4.6.1 Humidifier Overview

In one form of the present technology there is provided a humidifier 5000, as shown in FIG. 3b, that may comprise a water reservoir and a heating plate.

### 4.7 Glossary

For the purposes of the present technology disclosure, in certain forms of the present technology, one or more of the following definitions may apply. In other forms of the present technology, alternative definitions may apply.

### 4.7.1 General

Air: In certain forms of the present technology, air supplied to a patient may be atmospheric air, and in other forms of the present technology atmospheric air may be supplemented with oxygen.

Continuous Positive Airway Pressure (CPAP): CPAP treatment will be taken to mean the application of a supply of air or breathable gas to the entrance to the airways at a pressure that is continuously positive with respect to atmosphere, and preferably approximately constant through a respiratory cycle of a patient. In some forms, the pressure at the entrance to the airways will vary by a few centimeters of water within a single respiratory cycle, for example being higher during inhalation and lower during exhalation. In some forms, the pressure at the entrance to the airways will be slightly higher during exhalation, and slightly lower during inhalation. In some forms, the pressure will vary between different respiratory cycles of the patient, for example being increased in response to detection of indications of partial upper airway obstruction, and decreased in the absence of indications of partial upper airway obstruction.

### 4.7.2 Aspects of PAP Devices

Air circuit: A conduit or tube constructed and arranged in use to deliver a supply of air or breathable gas between a PAP device and a patient interface. In particular, the air circuit may be in fluid connection with the outlet of the pneumatic block and the patient interface. The air circuit may be referred to as air delivery tube. In some cases there may be separate limbs of the circuit for inhalation and exhalation. In other cases a single limb is used.

APAP: Automatic Positive Airway Pressure. Positive airway pressure that is continually adjustable between minimum and maximum limits, depending on the presence or absence of indications of SDB events.

Blower or flow generator: A device that delivers a flow of air at a pressure above ambient pressure.

Controller: A device, or portion of a device that adjusts an output based on an input. For example one form of controller has a variable that is under control—the control variable—that constitutes the input to the device. The output of the device is a function of the current value of the control variable, and a set point for the variable. A servo-ventilator may include a controller that has ventilation as an input, a target ventilation as the set point, and level of pressure support as an output. Other forms of input may be one or more of oxygen saturation (SaO₂), partial pressure of carbon dioxide (PCO₂), movement, a signal from a photoplethysmogram, and peak flow. The set point of the controller may be one or more of fixed, variable or learned. For example, the set point in a ventilator may be a long term average of the measured ventilation of a patient. Another ventilator may have a ventilation set point that changes with time. A pressure controller may be configured to control a blower or pump to deliver air at a particular pressure.

Therapy: Therapy in the present context may be one or more of positive pressure therapy, oxygen therapy, carbon dioxide therapy, control of dead space, and the administration of a drug.

Motor: A device for converting electrical energy into rotary movement of a member. In the present context the rotating member is an impeller, which rotates in place around a fixed axis so as to impart a pressure increase to air moving along the axis of rotation.

Positive Airway Pressure (PAP) device: A device for providing a supply of air at positive pressure to the airways.

Transducers: A device for converting one form of energy or signal into another. A transducer may be a sensor or detector for converting mechanical energy (such as movement) into an electrical signal. Examples of transducers include pressure sensors, flow sensors, carbon dioxide (CO₂) sensors, oxygen (O₂) sensors, effort sensors, movement sensors, noise sensors, a plethysmograph, and cameras.

Volute: The casing of the centrifugal pump that receives the air being pumped by the impeller, slowing down the flow rate of air and increasing the pressure. The cross-section of the volute increases in area towards the discharge port.

### 4.7.3 Aspects of the Respiratory Cycle

Apnea: Preferably, apnea will be said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate: The rate of spontaneous respiration of a patient, usually measured in breaths per minute.

Duty cycle: The ratio of inhalation time, Ti to total breath time, Ttot.

Effort (breathing): Preferably breathing effort will be said to be the work done by a spontaneously breathing person attempting to breathe.

Expiratory portion of a breathing cycle: The period from the start of expiratory flow to the start of inspiratory flow.

Flow limitation: Preferably, flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:

(i) Flattened: Having a rise followed by a relatively flat portion, followed by a fall.

(ii) M-shaped: Having two local peaks, one at the leading edge, and one at the trailing edge, and a relatively flat portion between the two peaks.

(iii) Chair-shaped: Having a single local peak, the peak being at the leading edge, followed by a relatively flat portion.

(iv) Reverse-chair shaped: Having a relatively flat portion followed by single local peak, the peak being at the trailing edge.

Hypopnea: Preferably, a hypopnea will be taken to be a reduction in flow, but not a cessation of flow. In one form, a hypopnea may be said to have occurred when there is a reduction in flow below a threshold for a duration. In one form in adults, the following either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal flow.

Inspiratory portion of a breathing cycle: Preferably the period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peak flow (Qpeak): The maximum value of flow during the inspiratory portion of the respiratory flow waveform.

Respiratory flow, airflow, patient airflow, respiratory airflow (Qr): These synonymous terms may be understood to refer to the PAP device's estimate of respiratory airflow, as opposed to “true respiratory flow” or “true respiratory airflow”, which is the actual respiratory flow experienced by the patient, usually expressed in litres per minute.

Tidal volume (VI): The volume of air inhaled or exhaled during normal breathing, when extra effort is not applied.

(inhalation) Time (Ti): The duration of the inspiratory portion of the respiratory flow waveform.

(exhalation) Time (Te): The duration of the expiratory portion of the respiratory flow waveform.

(total) Time (Ttot): The total duration between the start of the inspiratory portion of one respiratory flow waveform and the start of the inspiratory portion of the following respiratory flow waveform.

Typical recent ventilation: The value of ventilation around which recent values over some predetermined timescale tend to cluster, that is, a measure of the central tendency of the recent values of ventilation.

Upper airway obstruction (UAO): includes both partial and total upper airway obstruction. This may be associated with a state of flow limitation, in which the level of flow increases only slightly or may even decrease as the pressure difference across the upper airway increases (Starling resistor behaviour).

Ventilation (Vent): A measure of the total amount of gas being exchanged by the patient's respiratory system, including both inspiratory and expiratory flow, per unit time. When expressed as a volume per minute, this quantity is often referred to as “minute ventilation”. Minute ventilation is sometimes given simply as a volume, understood to be the volume per minute.

### 4.7.4 PAP Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow will be given the symbol Q. Total flow, Qt, is the flow of air leaving the PAP device. Vent flow, Qv, is the flow of air leaving a vent to allow washout of exhaled gases. Leak flow, Ql, is the flow rate of unintentional leak from a patient interface system. Respiratory flow, Qr, is the flow of air that is received into the patient's respiratory system.

Leak: Preferably, the word leak will be taken to be a flow of air to the ambient. Leak may be intentional, for example to allow for the washout of exhaled CO₂. Leak may be unintentional, for example, as the result of an incomplete seal between a mask and a patient's face.

Pressure: Force per unit area. Pressure may be measured in a range of units, including cmH₂O, g-f/cm², hectopascal. 1cmH₂O is equal to 1 g-f/cm² and is approximately 0.98 hectopascal. In this specification, unless otherwise stated, pressure is given in units of cmH₂O. For nasal CPAP treatment of OSA, a reference to treatment pressure is a reference to a pressure in the range of about 4-20 cmH₂O, or about 4-30 cmH₂O. The pressure in the patient interface is given the symbol Pm.

Sound Power: The energy per unit time carried by a sound wave. The sound power is proportional to the square of sound pressure multiplied by the area of the wavefront. Sound power is usually given in decibels SWL, that is, decibels relative to a reference power, normally taken as 10⁻¹² watt.

Sound Pressure: The local deviation from ambient pressure at a given time instant as a result of a sound wave travelling through a medium. Sound power is usually given in decibels SPL, that is, decibels relative to a reference power, normally taken as 20×10⁻⁶ pascal (Pa), considered the threshold of human hearing.

### 4.7.5 Terms for Ventilators

Adaptive Servo-Ventilator: A ventilator that has a changeable, rather than fixed target ventilation. The changeable target ventilation may be learned from some characteristic of the patient, for example, a respiratory characteristic of the patient.

Backup rate: A parameter of a ventilator that establishes the minimum respiration rate (typically in number of breaths per minute) that the ventilator will deliver to the patient, if not otherwise triggered.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

EPAP (or EEP): a base pressure, to which a pressure varying within the breath is added to produce the desired mask pressure which the ventilator will attempt to achieve at a given time.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the minimum value during expiration (e.g., PS=IPAP−EPAP). In some contexts pressure support means the difference which the ventilator aims to achieve, rather than what it actually achieves.

Servo-ventilator: A ventilator that measures patient ventilation has a target ventilation, and which adjusts the level of pressure support to bring the patient ventilation towards the target ventilation.

Spontaneous/Timed (S/T)—A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the respiratory portion of the breathing cycle by the patient's efforts.

Ventilator: A mechanical device that provides pressure support to a patient to perform some or all of the work of breathing.

### 4.7.6 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricula or Pinna: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfurt horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear) dividing the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramentale: The point of greatest concavity in the midline of the lower lip between labrale inferius and soft tissue pogonion

### 4.7.7 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.7.8 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.7.9 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, a preferred form of LSR has a Shore A (or Type A) indentation hardness in the range of about 35 to about 45 as measured using ASTM D2240.

Polycarbonate: a typically transparent thermoplastic polymer of Bisphenol-A Carbonate.

### 4.7.10 Aspects of a Patient Interface

Anti-asphyxia valve (AAV): The component or sub-assembly of a mask system that, by opening to atmosphere in a failsafe manner, reduces the risk of excessive CO₂ rebreathing by a patient.

Elbow: A conduit that directs an axis of flow of air to change direction through an angle. In one form, the angle may be approximately 90 degrees. In another form, the angle may be less than 90 degrees. The conduit may have an approximately circular cross-section. In another form the conduit may have an oval or rectangular cross-section.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. Preferably the headgear comprises a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to a mean portion of a patient interface having walls enclosing a volume of space, the volume having air therein pressurised above atmospheric pressure in use. A shell may form part of the walls of a mask plenum chamber. In one form, a region of the patient's face forms one of the walls of the plenum chamber.

Seal: The noun form (“a seal”) will be taken to mean a structure or barrier that intentionally resists the flow of air through the interface of two surfaces. The verb form (“to seal”) will be taken to mean to resist a flow of air.

Shell: A shell will preferably be taken to mean a curved structure having bending, tensile and compressive stiffness, for example, a portion of a mask that forms a curved structural wall of the mask. Preferably, compared to its overall dimensions it is relatively thin. In some forms, a shell may be faceted. Preferably such walls are airtight, although in some forms they may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel: (noun) A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. Preferably there is little or no leak flow of air from the swivel in use.

Tie: A tie will be taken to be a structural component designed to resist tension.

Vent: (noun) the structure that allows a deliberate controlled rate leak of air from an interior of the mask, or conduit to ambient air, to allow washout of exhaled carbon dioxide (CO₂) and supply of oxygen (O₂).

### 4.7.11 Terms Used in Relation to Patient Interface

Curvature (of a surface): A region of a surface having a saddle shape, which curves up in one direction and curves down in a different direction, will be said to have a negative curvature. A region of a surface having a dome shape, which curves the same way in two principle directions, will be said to have a positive curvature. A flat surface will be taken to have zero curvature.

Floppy: A quality of a material, structure or composite that is the combination of features of:
Readily conforming to finger pressure.
Unable to retain its shape when caused to support its own weight.
Not rigid.
Able to be stretched or bent elastically with little effort.

The quality of being floppy may have an associated direction, hence a particular material, structure or composite may be floppy in a first direction, but stiff or rigid in a second direction, for example a second direction that is orthogonal to the first direction.

Resilient: Able to deform substantially elastically, and to release substantially all of the energy upon unloading, within a relatively short period of time such as 1 second.

Rigid: Not readily deforming to finger pressure, and/or the tensions or loads typically encountered when setting up and maintaining a patient interface in sealing relationship with an entrance to a patient's airways.

Semi-rigid: means being sufficiently rigid to not substantially distort under the effects of mechanical forces typically applied during positive airway pressure therapy.

### 4.8 Other Remarks

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.9 REFERENCE SIGNS LIST

| | |
|---|---|
| packaging tray | 100 |
| cushion assembly region | 102 |
| stalk region | 103 |
| cushion assembly region | 104 |
| stalk region | 105 |
| cushion assembly region | 106 |
| stalk region | 107 |
| rotatable adapter region | 108 |
| tube surface | 110 |
| strap region | 112 |
| rigidiser arm region | 114 |
| tube region | 116 |
| flap | 118 |
| cutout | 119 |
| kink | 120 |
| hot tip gate | 122 |
| inner wall | 124 |
| outer wall | 126 |
| notch | 128 |
| notch | 129 |
| manual | 130 |
| rotatable adapter retaining rib | 132 |
| rib | 134 |
| frame retention post | 136 |
| support surface | 138 |
| patient | 1000 |
| bed partner | 1100 |
| patient interface system | 3000 |
| cushion assembly | 3002 |
| seal-forming structure | 3100 |
| plenum chamber | 3200 |
| structure | 3300 |
| right side strap portion | 3315 |
| left side strap portion | 3316 |
| back strap | 3317 |
| extension | 3350 |
| vent | 3400 |
| connection port | 3600 |
| pap device | 4000 |
| external housing | 4010 |
| upper portion | 4012 |
| lower portion | 4014 |
| panel | 4015 |
| chassis | 4016 |
| handle | 4018 |
| pneumatic block | 4020 |
| pneumatic component | 4100 |
| air filter | 4110 |
| inlet air filter | 4112 |
| outlet air filter | 4114 |
| blower | 4142 |
| air circuit | 4170 |
| short tube | 4180 |
| rotatable adapter | 4190 |
| electrical component | 4200 |
| PCBA | 4202 |
| electrical power supply | 4210 |
| input device | 4220 |
| humidifier | 5000 |

1. A packaging tray for a patient interface system adapted to provide respiratory therapy, the packaging tray comprising:
at least one cushion assembly region, each shaped to conform to at least a portion of a cushion assembly;
a tube region shaped to conform to at least a portion of a tube;
a positioning and stabilising structure region shaped to conform to at least a portion of a positioning and structure; and
a plurality of flaps adapted to retain documentation for the patient interface system.

2. The packaging tray of claim 1, wherein each of the plurality of flaps includes a kinked portion.

3. The packaging tray of claim 2, further comprising an outer wall, the plurality of flaps extending from the outer wall.

4. The packaging tray of claim 3, wherein the outer wall comprises at least one notch to allow access to the documentation retained by the plurality of flaps.

5. The packaging tray of claim 1, wherein each at least one cushion assembly region is arranged such that when cushion assemblies are placed in each at least one cushion assembly region, a seal-forming structure of each cushion assembly extends into a plenum chamber of an adjacent cushion assembly.

6. The packaging tray of claim 5, wherein each at least one cushion assembly region is further arranged such that when cushion assemblies are placed in each at least one cushion assembly region, each cushion assembly does not contact adjacent cushion assemblies.

7. The packaging tray of claim 1, wherein the positioning and stabilising structure region comprises a pair of rigidiser arm regions and a strap region.

8. The packaging tray of claim 7, wherein each of the pair of rigidiser arm regions is sloped to conform to a respective rigidiser arm of the patient interface system.

9. The packaging tray of claim 7, wherein the strap region is shaped and dimensioned to hold straps of a positioning and stabilising structure folded and/or tucked into the strap region.

10. The packaging tray of claim 1, further comprising an inner wall to separate the at least one tube region from the at least one cushion assembly region such that when the tube is placed in the packaging tray, at least a portion of the tube wraps around at least a portion of the inner wall.

11. The packaging tray of claim 1, further comprising a tube surface to support the tube above the positioning and stabilising structure.

12. The packaging tray of claim 1, further comprising a rotatable adapter region shaped to conform to at least a portion of a rotatable adapter.

13. The packaging tray of claim 1, further comprising polypropylene or high-impact polystyrene.

14. The packaging tray of claim 1, wherein the packaging tray is injection molded.

15. A method for packaging a patient interface system in a packaging tray, the method comprising:
inserting documentation into the packaging tray such that the documentation is retained against the packaging tray with a plurality of flaps of the packaging tray;
inserting cushion assemblies into respective cushion assembly regions of the packaging tray;
inserting a positioning and stabilising structure into a positioning and stabilising structure region of the packaging tray; and
inserting a tube into a tube region of the packaging tray.

16. The method of claim 15, wherein each cushion assembly is inserted into a respective cushion assembly region of the packaging tray such that a seal-forming structure of each cushion assembly extends into a plenum chamber of an adjacent cushion assembly.

17. The method of claim 15, further comprising inserting a rotatable adapter into a rotatable adapter region of the packaging tray.

18. The method of claim 15, wherein inserting the positioning and stabilising structure into the positioning and stabilising structure region of the packaging tray further comprises inserting rigidiser arms into respective rigidiser arm regions, the rigidiser arm regions shaped and dimensioned to substantially conform to the respective rigidiser arm.

19. The method of claim 15, inserting the positioning and stabilising structure into the positioning and stabilising structure region of the packaging tray further comprises folding and/or tucking a strap of the positioning and stabilising structure into a strap region of the packaging tray.

20. The method of claim 15, wherein inserting the tube into the tube region of the packaging tray further comprises at least partially wrapping the tube around an inner wall of the packaging tray and placing at least a portion of the tube on a tube surface of the packaging tray such that the tube is supported above the positioning and stabilising structure.

21. A packaging tray for a patient interface system adapted to provide respiratory therapy, the packaging tray comprising:
a plurality of cushion assembly regions, each shaped to conform to at least a portion of a cushion assembly;
a tube region shaped to conform to at least a portion of a tube; and
a positioning and stabilising structure region shaped to conform to at least a portion of a positioning and structure,
wherein the cushion assembly regions are shaped and dimensioned to retain and protect respective cushion assemblies in a nested arrangement in a fixed position.

22. The packaging tray of claim 21, wherein each one of the cushion assembly regions is shaped and dimensioned to retain and protect a different sized cushion assembly.

23. The packaging tray of claim 21, wherein the cushion assembly regions are arranged such that when the cushion assemblies are placed in the respective cushion assembly regions, a seal-forming structure of one of the cushion assemblies extends into a plenum chamber of an adjacent cushion assembly.

24. The packaging tray of claim 23, wherein the cushion assembly regions are further arranged such that when cushion assemblies are placed in each cushion assembly region, each cushion assembly does not contact adjacent cushion assemblies.

25. The packaging tray of claim 23, wherein the cushion assembly regions are further arranged such that when cushion assemblies are placed in each cushion assembly region, each cushion assembly contacts at least one adjacent cushion assembly.

26. The packaging tray of claim 25, wherein the seal-forming structure of each cushion assembly is not substantially deformed by contact with the adjacent cushion assembly.

27. The packaging tray of claim 21, wherein a seal-forming structure of each cushion assembly extends partially into a plenum chamber of an adjacent cushion assembly.

28. A packaging tray for a patient interface system adapted to provide respiratory therapy, the packaging tray comprising:
at least one cushion assembly region, each shaped to conform to at least a portion of a cushion assembly;
a tube region shaped to conform to at least a portion of a tube; and
a positioning and stabilising structure region shaped to conform to at least a portion of a positioning and structure,
wherein the packaging tray is about 178 mm long, about 116 mm wide, and about 38 mm in height.

29. A packaging tray for a patient interface system adapted to provide respiratory therapy, the packaging tray comprising:
at least one cushion assembly region, each shaped to conform to at least a portion of a cushion assembly;
a tube region shaped to conform to at least a portion of a tube; and
a positioning and stabilising structure region shaped to conform to at least a portion of a positioning and structure,
wherein about 85% to about 95% of an area of the packaging tray, when viewed from above, is adapted to retain and protect the patient interface system.

30. The packaging tray of claim 29, wherein the area is about 89.7%.

31. A packaging tray for a patient interface system adapted to provide respiratory therapy, the packaging tray comprising:
at least one cushion assembly region, each shaped to conform to at least a portion of a cushion assembly;
a tube region shaped to conform to at least a portion of a tube; and
a positioning and stabilising structure region shaped to conform to at least a portion of a positioning and structure,
wherein the packaging tray has a length-width ratio of approximately 1.53, a length-height ratio of approximately 4.68, and a width-height ratio of approximately 3.05.

32. A packaging tray for a patient interface system adapted to provide respiratory therapy, the packaging tray comprising:
three cushion assembly regions, each shaped to conform to at least a portion of a cushion assembly;
a tube region shaped to conform to at least a portion of a tube; and
a positioning and stabilising structure region shaped to conform to at least a portion of a positioning and structure,
wherein the cushion assembly regions are arranged such that each cushion assembly is oriented in a common direction when placed in respective cushion assembly regions.

33. A packaging tray for a patient interface system adapted to provide respiratory therapy, the packaging tray comprising:
at least one cushion assembly region, each shaped to conform to at least a portion of a cushion assembly;
a tube region shaped to conform to at least a portion of a tube; and
a positioning and stabilising structure region shaped to conform to at least a portion of a positioning and structure,
wherein at least about 80% of a volume of the packaging tray is utilized to protect and retain components of the patient interface system.

34. The packaging tray of claim 33, wherein about 87% to about 96% of the volume of the packaging tray is utilized to protect and retain components of the patient interface system.

35. The packaging tray of claim 34, wherein about 90% to about 93% of the volume of the packaging tray is utilized to protect and retain components of the patient interface system.

36. The packaging tray of claim 35, wherein about 92% of the volume of the packaging tray is utilized to protect and retain components of the patient interface system.
